SEQUENCE LISTING (11 sequences)

SEQ ID NO: 1 - DNA, 876 bp, synthetic construct
  misc_feature 1..876: note = Description of Artificial Sequence: Synthetic polynucleotide
  source 1..876: mol_type = other DNA
atggtgagggagcagtacaccacagcaaccgagggaatctgcatcgagaggccagagaaccagtacgtgtataagatcggcatctacggctggcggaagagatgtctgtatctgttcgtgctgctgctgctgatcatcctggtggtgaatctggccctgaccatctggatcctgaaagtgatgtggttttccccagcaggaatgggacacctgtgcgtgacaaaggacggactgcggctggagggagagtctgagttcctgtttcccctgtatgccaaggagatccacagcagagtggatagctccctgctgctgcagtccacccagaacgtgacagtgaacgcaaggaatagcgagggagaggtgaccggcagactgaaggtcggccccaagatggtggaggtgcagaatcagcagttccagatcaactccaatgacggcaagcctctgtttacagtggatgagaaggaggtggtggtgggcaccgacaagctgagggtgacaggacctgagggcgccctgttcgagcactctgtggagaccccactggtgcgcgcagacccttttcaggatctgaggctggagagcccaacacgcagcctgtccatggacgcacccagaggcgtgcacatccaggcacacgcaggcaagatcgaggccctgagccagatggatatcctgttccactctagcgacggcatgctggtgctggatgccgagaccgtgtgcctgcctaagctggtgcagggcacatggggcccatctggctcctctcagagcctgtacgagatctgcgtgtgcccagatggcaagctgtatctgtccgtggccggcgtgtctaccacatgccaggagcacaaccacatctgtctgtga

SEQ ID NO: 2 - protein, 291 aa, Homo sapiens
MVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHNHICL

SEQ ID NO: 3 - DNA, 2225 bp, synthetic construct
  misc_feature 1..2225: note = Description of Artificial Sequence: Synthetic polynucleotide
  source 1..2225: mol_type = other DNA
ctgcgcgctcgctcgctcactgaggccgcccgggcaaagcccgggcgtcgggcgacctttggtcgcccggcctcagtgagcgagcgagcgcgcagagagggagtggggttaaccaattggcgcggccgcaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccggtaccaccatggtgagggagcagtacaccacagcaaccgagggaatctgcatcgagaggccagagaaccagtacgtgtataagatcggcatctacggctggcggaagagatgtctgtatctgttcgtgctgctgctgctgatcatcctggtggtgaatctggccctgaccatctggatcctgaaagtgatgtggttttccccagcaggaatgggacacctgtgcgtgacaaaggacggactgcggctggagggagagtctgagttcctgtttcccctgtatgccaaggagatccacagcagagtggatagctccctgctgctgcagtccacccagaacgtgacagtgaacgcaaggaatagcgagggagaggtgaccggcagactgaaggtcggccccaagatggtggaggtgcagaatcagcagttccagatcaactccaatgacggcaagcctctgtttacagtggatgagaaggaggtggtggtgggcaccgacaagctgagggtgacaggacctgagggcgccctgttcgagcactctgtggagaccccactggtgcgcgcagacccttttcaggatctgaggctggagagcccaacacgcagcctgtccatggacgcacccagaggcgtgcacatccaggcacacgcaggcaagatcgaggccctgagccagatggatatcctgttccactctagcgacggcatgctggtgctggatgccgagaccgtgtgcctgcctaagctggtgcagggcacatggggcccatctggctcctctcagagcctgtacgagatctgcgtgtgcccagatggcaagctgtatctgtccgtggccggcgtgtctaccacatgccaggagcacaaccacatctgtctgtgactcgagggccgcaataaaagatctttattttcattagatctgtgtgttggttttttgtgtgtcctgcaggggcgcgcctaatctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 4 - DNA, 792 bp, synthetic construct
  misc_feature 1..792: note = Description of Artificial Sequence: Synthetic polynucleotide
  source 1..792: mol_type = other DNA
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 5 - DNA, 148 bp, synthetic construct
  misc_feature 1..148: note = Description of Artificial Sequence: Synthetic polynucleotide
  source 1..148: mol_type = other DNA
aggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtaccc

SEQ ID NO: 6 - DNA, 53 bp, synthetic construct
  misc_feature 1..53: note = Description of Artificial Sequence: Synthetic oligonucleotide
  source 1..53: mol_type = other DNA
ggccgcaataaaagatctttattttcattagatctgtgtgttggttttttgtg

SEQ ID NO: 7 - DNA, 110 bp, synthetic construct
  misc_feature 1..110: note = Description of Artificial Sequence: Synthetic polynucleotide
  source 1..110: mol_type = other DNA
ctgcgcgctcgctcgctcactgaggccgcccgggcaaagcccgggcgtcgggcgacctttggtcgcccggcctcagtgagcgagcgagcgcgcagagagggagtggggtt

SEQ ID NO: 8 - DNA, 104 bp, synthetic construct
  misc_feature 1..104: note = Description of Artificial Sequence: Synthetic polynucleotide
  source 1..104: mol_type = other DNA
ccactccctctctgcgcgctcgctcgctcactgaggccgggcgaccaaaggtcgcccgacgcccgggctttgcccgggcggcctcagtgagcgagcgagcgcgc

SEQ ID NO: 9 - DNA, 876 bp, Homo sapiens
  source 1..876: mol_type = unassigned DNA
atggtgcgtgagcagtacactacagccacagaaggcatctgcatagagaggccagagaatcagtatgtctacaaaattggcatttatggctggagaaagcgctgtctctacttgtttgttcttcttttactcatcatcctcgttgtgaatttagctcttacaatttggattcttaaagtgatgtggttttctccagcaggaatgggccacttgtgtgtaacaaaagatggactgcgcttggaaggggaatcagaatttttattcccattgtatgccaaagaaatacactccagagtggactcatctctgctgctacaatcaacccagaatgtgactgtaaatgcgcgcaactcagaaggggaggtcacaggcaggttaaaagtcggtcccaaaatggtagaagtccagaatcaacagtttcagatcaactccaacgacggcaagccactatttactgtagatgagaaggaagttgtggttggtacagataaacttcgagtaactgggcctgaaggggctctttttgaacattcagtggagacaccccttgtcagagccgacccgtttcaagaccttagattagaatcccccactcggagtctaagcatggatgccccaaggggtgtgcatattcaagctcacgctgggaaaattgaggcgctttctcaaatggatattctttttcatagtagtgatggaatgcttgtgcttgatgctgaaactgtgtgcttacccaagctggtgcaggggacgtggggtccctctggcagctcacagagcctctacgaaatctgtgtgtgtccagatgggaagctgtacctgtctgtggccggtgtgagcaccacgtgccaggagcacagccacatctgcctctga

SEQ ID NO: 10 - protein, 535 aa, synthetic construct
  REGION 1..535: note = Description of Artificial Sequence: Synthetic polypeptide
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 11 - DNA, 2217 bp, synthetic construct
  misc_feature 1..2217: note = Description of Artificial Sequence: Synthetic polynucleotide
  source 1..2217: mol_type = other DNA
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

(19) United States
(12) Patent Application Publication
(10) Pub. No.: US 2026/0061073 A1
(43) Pub. Date: Mar. 5, 2026

(54) GENE THERAPY FOR LIMB-GIRDLE MUSCULAR DYSTROPHY TYPE 2C

(71) Applicant: RESEARCH INSTITUTE AT NATIONWIDE CHILDREN'S HOSPITAL, Columbus, OH (US)

(72) Inventor: Louise R. RODINO-KLAPAC, Columbus, OH (US)

(73) Assignee: RESEARCH INSTITUTE AT NATIONWIDE CHILDREN'S HOSPITAL, Columbus, OH (US)

(21) Appl. No.: 19/065,933

(22) Filed: Feb. 27, 2025

Related U.S. Application Data
(63) Continuation of application No. 16/966,407, now Pat. No. 12,263,230.
(60) Provisional application No. 62/624,616, filed on Jan. 31, 2018.

(51) Int. Cl. A61K 48/00; A61K 45/06; A61P 21/00; C07K 14/47; C12N 15/86

(52) U.S. Cl. CPC A61K 48/005; A61K 48/0075; A61P 21/00; C07K 14/4707; C12N 15/86; A61K 45/06

(57) ABSTRACT

The disclosure relates to gene therapy vectors, such as AAV vectors, comprising a polynucleotide encoding γ-sarcoglycan (SGCG) and methods of using such gene therapy vectors to treat subjects suffering from a muscular dystrophy. e.g. limb girdle dystrophy type 2C (LGMD2C).

### CROSS-REFERENCE TO RELATED PATENT APPLICATION

This application is a continuation of U.S. patent application Ser. No. 16/966,407, filed Jul. 30, 2020, now U.S. Pat. No. 12,263,230, which is a is a U.S. national stage application under 35 U.S.C. § 371 of International Application No. PCT/US2019/015779, filed Jan. 30, 2019, which, claims priority under 35 U.S.C. § 119 (c) to U.S. Provisional Patent Application No. 62/624,616, filed Jan. 31, 2018, the contents of each of which are hereby incorporated by reference in their entirety.

### SEQUENCE LISTING

The instant application contains a Sequence Listing which has been submitted electronically in XML format and is hereby incorporated by reference in its entirety. Said XML copy, created on Jul. 31, 2025, is named 140056-0811_SL.xml and is 22,579 bytes in size.

### FIELD OF THE INVENTION

The invention relates to gene therapy. More specifically, the disclosure provides gene therapy vectors such as adeno-associated virus (AAV) vectors for treating muscular dystrophy, e.g. limb girdle dystrophy type 2C (LGMD2C).

### BACKGROUND OF THE INVENTION

Muscular dystrophies (MDs) are a group of genetic diseases. The group is characterized by progressive weakness and degeneration of skeletal muscles that control movement. Some forms of MD develop in infancy or childhood, while others may not appear until middle age or later. The disorders differ in terms of the distribution and extent of muscle weakness—some forms of MD also affect cardiac muscle, the age of onset, the rate of progression, and the pattern of inheritance.

One group of MDs is the limb girdle group (LGMD) of MDs. LGMDs are rare conditions, which present differently in different people with respect to age of onset, areas of muscle weakness, heart and respiratory involvement, rate of progression and severity. LGMDs can begin in childhood, adolescence, young adulthood or even later. Both genders are affected equally. LGMDs cause weakness in the shoulder and pelvic girdle, with nearby muscles in the upper legs and arms sometimes also weakening with time. Weakness of the legs often appears before that of the arms. Facial muscles are usually unaffected. As the condition progresses, affected individuals can develop problems with walking and may need to use a wheelchair over time. The involvement of shoulder and arm muscles can lead to difficulty in raising arms over head and in lifting objects. In some types of LGMD, the heart and breathing muscles may be involved.

LGMD2C (limb girdle dystrophy type 2C) is caused by gamma (γ)-sarcoglycan (SGCG) deficiency. Like the other sarcoglycanopathies, it presents as a progressive muscular dystrophy starting in the girdle muscles before extending to lower and finally upper extremity muscles. Presentation typically occurs in mid to late teens. In attempting to treat LGMD2C, no form of drug therapy, including even corticosteroids, has changed the course of the disease.

Functional improvement in patients suffering from LGMD2C and other muscular dystrophies require both gene restoration and reduction of fibrosis. There is a need in the art for compositions and methods for treating LGMD2C and other muscular dystrophies.

### SUMMARY OF THE INVENTION

Described herein are gene therapy vectors, e.g. recombinant adeno-associated virus (AAV) vectors, encoding γ-sarcoglycan and methods of delivering such vectors encoding γ-sarcoglycan to the muscle to reduce or prevent fibrosis; to maintain or improve muscle function; to increase muscular force; to increase muscle endurance; or to treat a γ-sarcoglycanopathy in a mammalian subject suffering from muscular dystrophy.

In addition, the disclosure provides therapies and approaches using gene therapy vectors to deliver γ-sarcoglycan to address the gene defect observed in LGMD2C (limb girdle dystrophy type 2C). In one aspect provided herein is a method for one or more of treating γ-sarcoglycanopathy; increasing muscular force, muscle endurance, and/or muscle mass; reducing fibrosis; reducing contraction-induced injury; decreasing fatty infiltration; and/or decreasing central nucleation in a subject in need thereof, and/or treating muscular dystrophy reducing degenerating fibers or necrotic fibers; reducing inflammation; elevating creatine kinase levels; treating myofiber atrophy and hypertrophy, and/or decreasing dystrophic calcification in a subject suffering from muscular dystrophy, the methods comprising, or consisting essentially of, or yet further consisting of administering to the subject a therapeutically effective amount of a recombinant adeno-associated virus (AAV) vector, wherein the rAAV vector comprises, or consists essentially of, or yet further consists of a gene expression cassette that comprises, or consists essentially of, or yet further consists of a polynucleotide sequence encoding γ-sarcoglycan under the transcriptional control of a promoter, said cassette flanked by one or more AAV inverted terminal repeats.

In one aspect, described herein is a recombinant AAV (rAAV) vector comprising, or consisting essentially of, or yet further consisting of a polynucleotide sequence encoding γ-sarcoglycan under the transcriptional control of a promoter. In some embodiments, the polynucleotide sequence encoding γ-sarcoglycan comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 1 and encodes a protein that retains γ-sarcoglycan activity. In some embodiments, the polynucleotide sequence encoding γ-sarcoglycan comprises, or consists essentially of, or yet further consists of the nucleotide sequence set forth in SEQ ID NO: 1. In some embodiments, the polynucleotide sequence encoding γ-sarcoglycan consists the nucleotide sequence set forth in SEQ ID NO: 1 or a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 1 and encodes a protein that retains γ-sarcoglycan activity, that in one aspect, retains the nucleotide changes of SEQ ID NO: 1 as compared to the corresponding nucleotides in wild-type human polynucleotide encoding γ-sarcoglycan

In another aspect, an rAAV vector described herein comprises, or consists essentially of, or yet further consists of a polynucleotide sequence encoding γ-sarcoglycan that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the amino acid sequence of SEQ ID NO: 2, and the protein retains γ-sarcoglycan activity.

γ-sarcoglycan activity is critical for muscle function. γ-sarcoglycan is one of several sarcolemmal transmembrane glycoproteins that interact with dystrophin and forms the dystrophin-glycoprotein complex, which spans the sarcolemma and is comprised of dystrophin, syntrophin, α-dystroglycans and β-dystroglycans, and sarcoglycans including γ-sarcoglycan. The dystrophin-glycoprotein complex provides a structural link between the subsarcolemmal cytoskeleton and the extracellular matrix of muscle cells. Non-limiting examples of muscle cells include cardiac, diaphragm, leg, pelvic girdle, shoulder and arm muscle cells. Further non-limiting examples of γ-sarcoglycan activity and consequences of γ-sarcoglycanopathy are described in Blake et al. (2002) Physiol Rev.; 82 (2): 291-329 and Tarakci et al. (2016) Front Biosci (Landmark Ed); 21:744-56.

In another aspect, the rAAV vectors described herein may be operably linked to a promoter and/or a muscle-specific control element to restrict expression to muscle. For example the muscle-specific control element is human skeletal actin gene element (GenBank Accession No. NG_006672.1), cardiac actin gene element (GenBank Accession No. NG_007553.1), myocyte-specific enhancer binding factor MEF (GenBank Accession No. NG_016443.2), muscle creatine kinase (MCK) (GenBank Accession No. AF188002.1), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE).

In some embodiments, the muscle-specific promoter is MHCK7 (SEQ ID NO: 4) or an equivalent thereof. An exemplary rAAV vector described herein is pAAV.MHCK7.hSCGC which comprises, or consists essentially of, or yet further consists of the nucleotide sequence of SEQ ID NO: 3 or an equivalent thereof; wherein the MHCK7 promoter spans nucleotides 136-927, a CMV intron spans nucleotides 937-1084, the γ-sarcoglycan sequence spans nucleotides 1094-1968 and the polyA spans nucleotides 1976-2028. In certain cases, pAAV.MHCK7.hSCGC is packaged in an AAV rh74 capsid.

The AAV can be any serotype, for example AAV1, AAV2, AAV3, AAV4, AAV5, AAV6, AAV7, AAV8, AAV9, AAV-10, AAV-11, AAV-12, AAV-13 or AAV rh74. In some embodiments, a rAAV vector comprises the inverted terminal repeat (ITR) sequences of AAV2.

Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014).

Compositions comprising or consisting essentially of any of the rAAV vectors described herein are also contemplated.

Methods of producing a recombinant AAV vector particle comprising culturing a cell that has been transfected with any recombinant AAV vector described herein and recovering recombinant AAV particles from the supernatant of the transfected cells are also provided. Viral particles comprising or consisting essentially of any of the recombinant AAV vectors described herein are also contemplated.

Methods of reducing fibrosis in a mammalian subject in need thereof is also provided. In this regard, the method comprises, or consists essentially of, or yet further consists of administering a therapeutically effective amount of an AAV vector described herein (or composition comprising, or consisting essentially of an AAV vector described herein) to the mammalian subject. In some embodiments, the mammalian subject suffers from muscular dystrophy. In some embodiments, administration of an AAV vector described herein (or composition comprising, or consisting essentially of an AAV vector described herein) reduces fibrosis in skeletal muscle or in cardiac muscle of the subject.

In another aspect, described herein is a method of increasing muscular force or muscle mass or muscle endurance in a mammalian subject comprising, or consisting essentially of, or yet further consisting of administering a therapeutically effective amount of an AAV vector described herein (or composition comprising, or consisting essentially of an AAV vector described herein) to the mammalian subject.

In any of the methods of the disclosure, the subject may be suffering from muscular dystrophy such as limb-girdle muscular dystrophy or any other dystrophin-associated muscular dystrophy.

Also provided is a method of treating muscular dystrophy in a mammalian subject comprising, or consisting essentially of, or yet further consisting of administering a therapeutically effective amount of an AAV vector described herein (or composition comprising, or consisting essentially of an AAV vector described herein) to the mammalian subject. In some embodiments, the muscular dystrophy is limb-girdle muscular dystrophy.

In any of the methods of the disclosure, the rAAV is administered by any appropriate mode of administration, e.g., intramuscular injection or intravenous injection. In addition, in any of the method of the disclosure, the rAAV is administered systemically, such as parental administration by injection, infusion or implantation.

The compositions of the disclosure are formulated for intramuscular injection or intravenous injection. In addition, the compositions of the disclosure are formulated for systemic administration, such as parental administration by injection, infusion or implantation.

In addition, any of the compositions formulated for administration to a subject suffering from muscular dystrophy (such as limb-girdle muscular dystrophy or any other dystrophin-associated muscular dystrophy). Also described herein are combination therapies comprising, or consisting essentially of one or more of the compositions disclosed herein and a corticosteroid. Provided herein are host cells comprising the rAAV vector of this disclosure. Further provided herein are kits comprising any of one or more of the embodiments disclosed herein and instructions for use. The kits can comprise, or consist essentially of, one or more of the compositions disclosed herein and a corticosteroid or one or more of the combination therapies provided herein. In any of the uses of the disclosure, the medicament is formulated for administration, e.g., intramuscular injection or intravenous injection. In addition, in any of the uses of the disclosure, the medicament is formulated for systemic administration, such as parental administration by injection, infusion or implantation. In addition, any of the medicaments may be prepared for administration to a subject suffering from muscular dystrophy (such as limb-girdle muscular dystrophy or any other dystrophin associated muscular dystrophy).

The foregoing paragraphs are not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. The invention includes, as an additional aspect, all embodiments of the invention narrower in scope in any way than the variations defined by specific paragraphs above. For example, where certain aspects of the invention that are described as a genus, it should be understood that every member of a genus is, individually, an aspect of the invention.

### BRIEF DESCRIPTION OF THE DRAWINGS

FIG. 1 depicts an AAV vector (scAAVrh74.MHCK7.hSGCG) comprising a codon-optimized full-length human γ-sarcoglycan (hSCGB) cDNA (SEQ ID NO: 1). The construct is flanked by two ˜100 bp AAV inverted terminal repeats (ITR), includes a codon optimized human γ-sarcoglycan cDNA (hSGCG), chimeric intron (Intron), synthetic polyadenylation signal (pA), and is driven by the skeletal and cardiac muscle specific MHCK7 promoter.

FIG. 2 depicts Hematoxylin and Eosin (H&E) staining of tibialis anterior (TA) muscle from 8 week old BL6 WT mice and γ-sarcoglycan knockout (γ-SG KO) mice showing a dystrophic phenotype in diseased mice.

FIGS. 3A-3C depict in vivo vector potency. scAAVrh74.MHCK7. hSGCG was injected into the tibialis anterior (TA) muscle of γ-SG KO mice at 3e10 vg total dose. FIG. 3A shows immunofluorescence staining of TA muscle in γ-SG KO mice. Nearly 100% γ-sarcoglycan protein expression at the sarcolemma resulted from vector delivery. FIG. 3B shows a Western blot for γ-sarcoglycan expression in injected TA muscles from treated mice #794, 795. FIG. 3C shows immunofluorescence staining of TA muscle in control wild-type mice (“BL6 WT TA”) or uninjected control γ-SG KO mice (“GSG KO TA”), as well as an unstained sample (“GSG KO No Primary”).

FIGS. 4A-4B depict in vivo vector potency and toxicity in BL6 wild-type (WT) mice. FIG. 4A shows immunofluorescence staining indicating overexpression of γ-sarcoglycan by membrane and intracellular staining. FIG. 4B shows Western blotting indicating overexpression of γ-sarcoglycan in injected LTA muscle.

FIG. 5 depicts in vivo vector potency and toxicity in BL6 wild-type (WT) mice. H&E staining of uninjected and injected BL6 WT TA muscles shows no toxicity with complete absence of any central nuclei, necrotic fibers, inflammatory infiltration, or fibrotic tissue.

FIG. 6 depicts immunofluorescence staining for γ-sarcoglycan on TA, gastrocnemius (GAS), quadriceps (QUAD), gluteus (GLUT), PSOAS, TRICEP, diaphragm, and heart muscle, demonstrating widespread expression of γ-sarcoglycan.

FIG. 7 depicts immunofluorescence staining of IV potency tissues. IF staining for γ-sarcoglycan in various skeletal muscles, diaphragm, and heart demonstrates robust expression with rare negative fibers 6 weeks following systemic delivery of scAAVrh.74.MHCK7.hSGCG.

FIGS. 8A-8B show expression of SGCG in IV treated animals. Immunofluorescence imaging of skeletal muscles, diaphragm, and heart from SGCG−/− mice intravenously injected with 1e13 vg total dose scAAVrh.74.MHCK7.hSGCG is shown in representative 20× images (FIG. 8A). Western blotting shows hSGCG expression in all skeletal muscles and the heart from mice given intravenous delivery of scAAVrh.74.MHCK7.hSGCG (FIG. 8B).

FIGS. 9A-9B show histological evaluation of tissues following systemic treatment. Hematoxylin & Eosin staining of TRI and DIA skeletal muscles in BL6 WT, untreated SGCG−/−, and AAV.MHCK7.hSGCG treated SGCG−/− mice shows the reversal of dystrophic pathology following treatment (FIG. 9A). Quantification of the percentage of fibers with central nucleation shows a decrease in treated muscles. BL6 WT (n=5), untreated SGCG−/− (n=6), AAV.MHCK7.hSGCG treated (n=5) (FIG. 9B). ***=p<0.001, ***=p<0.0001.

FIGS. 10A-10F show fiber diameter quantification. Quantification of fiber diameters was performed in the GAS (FIG. 10A), PSOAS (FIG. 10B), and TRI (FIG. 10C) of BL6 WT (n=5), untreated SGCG−/− (n=6), and AAV.MHCK7.hSGCG treated SGCG−/− (n=5) mice and normalization of fiber diameter distribution following treatment. Average fiber diameter is decreased in in the GAS (FIG. 10D), PSOAS (FIG. 10E), and TRI (FIG. 10F) muscle of untreated SGCG−/− mice and increased to WT levels in each muscle following AAV.MHCK7.hSGCG treatment in SGCG−/− mice. ****=p<0.0001.

FIGS. 11A-11C show TA and diaphragm physiology. TA and DIA muscles of BL6 WT (n=5), untreated SGCG−/− (n=6), and AAV.MHCK7.hSGCG treated (n=5) mice subjected to measurement of normalized specific force production. TA muscle subjected to eccentric contraction injury protocol (FIG. 11A). Improvement in TA specific force output and resistance to contraction induced injury in treated SGCG−/− mice was observed (FIG. 11B). DIA specific force output was restored to WT levels in treated SGCG−/− mice (FIG. 11C). *=p<0.05, ****=p<0.0001.

FIG. 12 shows laser monitoring of open-field cage activity. Overall ambulation in x and y planes is decreased in SGCG−/− mice and improved in AAV.MCHK7.hSGCG treated mice. BL6 WT (n=6), untreated SGCG−/− (n=6), and AAV.MHCK7.hSGCG treated (n=5).

FIG. 13 shows biodistribution of vector genomes. Vector genome distribution of average vg copies per microgram genomic DNA (gDNA) were measured in various tissues from two SGCG−/− mice 3 months after IV delivery of 1e13 vg total dose of scAAVrh.74.MHCK7.hSGCG.

FIGS. 14A-14B show comparison of serum ALT and AST. Serum from BL6 WT mice (n=6), untreated SGCG−/− mice (n=6), and AAV.MHCK7.hSGCG IV treated SGCG−/− mice (n=5) (1e13 vg total dose) was analyzed for biochemical component levels. Liver enzymes alkaline aminotransferase (ALT, FIG. 14A) and aspartate aminotransferase (AST, FIG. 14B) were elevated in diseased SGCG−/− mice and returned to near WT levels following treatment. *=p<0.05. Dashed lines represent lower and upper limits of normal range.

### DETAILED DESCRIPTION

The present disclosure relates to administration of a recombinant adeno-associated virus (rAAV) vector comprising a polynucleotide expressing γ-sarcoglycan for a reduction or complete reversal of muscle fibrosis in an individual suffering from limb-girdle muscular dystrophy As demonstrated in the Examples, administration of the rAAV vector described herein resulted in restoration of γ-sarcoglycan expression in knockout mice. Administration of an rAAV vector described herein will result in the reversal of dystrophic features including fewer degenerating fibers, reduced inflammation, and improved functional recovery by protection against eccentric contraction with increased force generation. The disclosure encompasses treatment of limb-girdle muscular dystrophy in a subject (e.g., a human subject) by administration of a rAAV vector described herein.

In the present description, any concentration range, percentage range, ratio range, or integer range is to be understood to include the value of any integer within the recited range and, when appropriate, fractions thereof (such as one tenth and one hundredth of an integer) unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously. As used herein, “plurality” may refer to one or more components (e.g., one or more miRNA target sequences). In this application, the use of “or” means “and/or” unless stated otherwise.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, 5, 6, 7, 8, 9, 10, 15, 20, 25, 30, 35, 40, 45, 50, 55, 60, 65, 70, 75, 80, 85, 90, 95, 99 or 100% as compared to a reference value. A decrease or reduction in a particular value may also be represented as a fold-change in the value compared to a reference value, for example, at least 1, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, 5, 6, 7, 8, 9, 10, 15, 20, 25, 30, 35, 40, 45, 50, 55, 60, 65, 70, 75, 80, 85, 90, 95, 99, 100, 200, 300, 400, 500% or more as compared to a reference value. An increase in a particular value may also be represented as a fold-change in the value compared to a reference value, for example, at least 1-fold, 2, 3, 4, 5, 6, 7, 8, 9, 10, 15, 20, 30, 40, 50, 60, 70, 80, 90, 100, 200, 500, 1000-fold or more, increase as compared to the level of a reference value.

“Complementary” refers to the capacity for pairing, through base stacking and specific hydrogen bonding, between two sequences comprising naturally or non-naturally occurring (e.g., modified as described above) bases (nucleosides) or analogs thereof. For example, if a base at one position of a nucleic acid is capable of hydrogen bonding with a base at the corresponding position of a target, then the bases are considered to be complementary to each other at that position. Nucleic acids can comprise universal bases, or inert abasic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject.

“Treating” as used herein refers to delivering an agent or composition to a subject to affect a physiologic outcome. In some embodiments, treating refers to the treatment of a disease in a subject, e.g., in a human, including (a) inhibiting the disease, e.g., arresting disease development or preventing disease progression; (b) relieving the disease, e.g., causing regression of the disease state; (c) curing the disease; and (d) preventing onset of disease, e.g., arresting disease development in an asymptomatic subject identified as a carrier of a genetic defect. In one aspect, treatment excludes prophylaxis or prevention.

When the disease is muscular dystrophy the following clinical end points are non-limiting examples of treatment: decrease in specific force, increase in resistance to injury, increase in muscular force, increase in muscle endurance, increase in muscle mass, reduction in contraction-induced injury, decrease in fatty infiltration, decrease in central nucleation, reduction in degenerating fibers or necrotic fibers, reduction in inflammation, elevation in creatine kinase levels, decrease in myofiber atrophy and hypertrophy and/or decrease in dystrophic calcification.

When the disease is fibrosis, the following clinical end points are non-limiting examples of treatment: reduction in fibrotic tissue, reduction in inflammation, reduction in fibroblastic lesions, reduction in activated fibroblast proliferation, reduction in myofibroblast genesis, reduction in rate of decline of Forced Vital Capacity (FVC), wherein FVC is the total amount of air exhaled during the lung function test, absolute and relative increases from baseline in FVC, absolute increase from baseline in FVC (% Predicted), increase in progression-free survival time, decrease from baseline in St George's Respiratory Questionnaire (SGRQ) total score, wherein SGRQ is a health-related quality of life questionnaire divided into 3 components: symptoms, activity and impact and the total score (summed weights) can range from 0 to 100 with a lower score denoting a better health status, and relative decrease from baseline in high resolution computerized tomography (HRCT) quantitative lung fibrosis (QLF) score, wherein the QLF score ranges from 0 to 100% and greater values represent a greater amount of lung fibrosis and are considered a worse health status. Non-limiting examples clinical end points for fibrosis treatment and tests that can be performed to measure said clinical end points are described in the following clinical trials: NCT03733444 (clinicaltrials.gov/ct2/show/NCT03733444) (last accessed on Jan. 9, 2019), NCT00287729 (clinicaltrials.gov/ct2/show/NCT00287729) (last accessed on Jan. 9, 2019), NCT00287716 (clinicaltrials.gov/ct2/show/NCT00287716) (last accessed on Jan. 9, 2019), NCT02503657 (clinicaltrials.gov/ct2/show/NCT02503657) (last accessed on Jan. 9, 2019), NCT00047645 (clinicaltrials.gov/ct2/show/NCT00047645) (last accessed on Jan. 9, 2019), NCT02802345 (clinicaltrials.gov/ct2/show/NCT02802345) (last accessed on Jan. 9, 2019), NCT01979952 (clinicaltrials.gov/ct2/show/NCT01979952) (last accessed on Jan. 9, 2019), NCT00650091 (clinicaltrials.gov/ct2/show/NCT00650091) (last accessed on Jan. 9, 2019), NCT01335464 (clinicaltrials.gov/ct2/show/NCT01335464) (last accessed on Jan. 9, 2019), NCT01335477 (clinicaltrials.gov/ct2/show/NCT01335477) (last accessed on Jan. 9, 2019), NCT01366209 (clinicaltrials.gov/ct2/show/NCT01366209) (last accessed on Jan. 9, 2019). Further non-limiting examples clinical end points for fibrosis treatment and tests that can be performed to measure said clinical end points are described in King et al, (2014) N Engl J Med. May 29; 370 (22): 2083-92 and Richeldi et al., (2014) N Engl J Med. May 29; 370 (22): 2071-82.

The term “effective amount” or “therapeutically effective amount” refer to the minimum amount of an agent or composition required to result in a particular physiological effect (e.g., an amount required to increase, activate, or enhance a particular physiological effect). The effective amount or therapeutically effective amount of a particular agent may be represented in a variety of ways based on the nature of the agent, such as mass/volume, #of cells/volume, particles/volume, (mass of the agent)/(mass of the subject), #of cells/(mass of subject), or particles/(mass of subject). The effective amount or therapeutically effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans and/or domestic animals.

As used herein “vector” refers to a nucleic acid molecule capable transferring or transporting a nucleic acid molecule to cell, along with, in a viral vector, one or more viral proteins, such as for encapsulated viruses the capsid of the virus. The transferred nucleic acid is generally linked to, e.g., inserted into, the vector nucleic acid molecule. A vector may include sequences that direct autonomous replication or reverse transcription in a cell, or may include sequences sufficient to allow integration into host cell DNA. “Vectors” include gene therapy vectors. As used herein, the term “gene therapy vector” refers to a vector capable of use in performing gene therapy, e.g., delivering a polynucleotide sequence encoding a therapeutic polypeptide to a subject. Gene therapy vectors may comprise a polynucleotide (“transgene”) encoding a protein, e.g., γ-sarcoglycan.

As used herein, the term “expression cassette” refers to a DNA segment that is capable in an appropriate setting of driving the expression of a polynucleotide (e.g., a transgene) encoding a protein (e.g., γ-sarcoglycan) that is incorporated in said expression cassette. When introduced into a host cell, an expression cassette inter alia is capable of directing the cell's machinery to transcribe the transgene into RNA, which is then usually further processed and finally translated into the therapeutically active polypeptide. The gene therapy vector can comprise, or consist essentially of expression cassette. The term expression cassette excludes polynucleotide sequences 5′ to the 5′ ITR and 3′ to the 3′ ITR. Provided herein are host cells comprising, or consisting essentially of, or yet further consisting of the rAAV vector of this disclosure. The cells can be of any appropriate species, e.g., mammalian cells.

As used herein, the phrases “operably linked” or “under the transcriptional control” with respect to a polynucleotide refers, interchangeably, to a configuration of promoter or muscle-specific control element and polynucleotide that enables the polynucleotide to be transcribed by a polymerase capable of binding to the promoter. In one aspect, the muscle-specific control element is to restrict expression to muscle. Non-limiting examples of muscle-specific control elements are human skeletal actin gene element (GenBank Accession No. NG_006672.1), cardiac actin gene element (GenBank Accession No. NG_007553.1), myocyte-specific enhancer binding factor MEF (GenBank Accession No. NG_016443.2), muscle creatine kinase (MCK) (GenBank Accession No. AF188002.1), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

As used herein, the term “AAV” is a standard abbreviation for adeno-associated virus. Adeno-associated virus is a single-stranded DNA parvovirus that grows only in cells in which certain functions are provided by a co-infecting helper virus. General information and reviews of AAV can be found in, for example, Carter, 1989, Handbook of Parvoviruses, Vol. 1, pp. 169-228, and Berns, 1990, Virology, pp. 1743-1764, Raven Press, (New York). It is fully expected that the same principles described in these reviews will be applicable to additional AAV serotypes characterized after the publication dates of the reviews because it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including two 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 {1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is inserted as cloned DNA in plasmids, which makes construction of recombinant genomes feasible. Furthermore, because the signals directing AAV replication and genome encapsidation are contained within the ITRs of the AAV genome, some or all of the internal approximately 4.3 kb of the genome (encoding replication and structural capsid proteins, rep-cap) may be replaced with foreign DNA. To generate AAV vectors, the rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics. Recombinant AAV (rAAV) genomes of the disclosure comprise, or consist essentially of, or yet further consist of a nucleic acid molecule encoding γ-sarcoglycan (e.g., SEQ ID NO: 1) and one or more AAV ITRs flanking the nucleic acid molecule. AAV DNA in the rAAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh74. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). The nucleotide sequences of the genomes of various AAV serotypes are known in the art. To promote skeletal muscle specific expression, AAV1, AAV5, AAV6, AAV8 or AAV9 may be used. Thus, in one aspect, described herein is a recombinant AAV vector comprising, or consisting essentially of a polynucleotide sequence encoding γ-sarcoglycan under the transcriptional control of a promoter and/or a muscle-specific control element. In some embodiments, the polynucleotide sequence encoding γ-sarcoglycan comprises, or consists essentially of, or yet further consists of a sequence e.g. at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence of codon-optimized human γ-sarcoglycan, which is set forth in SEQ ID NO: 1 (see Table 1) and encodes protein that retains γ-sarcoglycan activity. In some embodiments, the polynucleotide sequence encoding γ-sarcoglycan comprises the nucleotide sequence set forth in SEQ ID NO: 1 or a sequence e.g. at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% identical to SEQ ID NO. 1 that encodes a protein that retains γ-sarcoglycan activity.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared. A polynucleotide or polynucleotide region (or a polypeptide or polypeptide region) having a certain percentage (for example, 60%, 65%, 70%, 75%, 80%, 85%, 90%, 95%, 98% or 99%) of “sequence identity” to a reference sequence means that, when aligned, that percentage of bases (or amino acids) at each position in the test sequence are identical to the base (or amino acid) at the same position in the reference sequence. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/blast/Blast.cgi. An “equivalent” of a polypeptide or protein is one that has a certain sequence identity to that reference polypeptide or protein, (for example, 60%, 65%, 70%, 75%, 80%, 85%, 90%, 95%, 98% or 99% identity to the reference) and retains similar activity or function compared to the reference polypeptide or protein.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

In one aspect, the gene expression cassette of rAAV vector of this disclosure is flanked by one or more AAV inverted terminal repeats. In another aspect, the polynucleotide sequence encoding γ-sarcoglycan of rAAV vector comprises, or consists essentially of, or yet further consists of a nucleotide sequence at least 95% identical to SEQ ID NO: 1 and/or the nucleotide sequence set forth in SEQ ID NO: 1 and encodes protein that retains γ-sarcoglycan activity. In a further aspect, the polynucleotide sequence encoding γ-sarcoglycan of rAAV vector encodes an amino acid sequence at least 95% identical, at least 99% identical, or 100% to SEQ ID NO: 2 and encodes protein that retains γ-sarcoglycan activity.

In some embodiments, the rAAV vector disclosed herein is of the serotype AAV1, AAV2, AAV4, AAV5, AAV6, AAV7, AAV8, AAV9, AAV10, AAV11, AAV12, AAV13 or AAV rh74. In other embodiments, the genome of the rAAV vector comprises, or consists essentially of a muscle-specific control element and wherein muscle-specific control element is operably linked to the polynucleotide sequence. Non-limiting examples of muscle-specific control elements are human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin I gene element, hypoxia-inducible nuclear factors, steroid-inducible element, and glucocorticoid response element (gre). In one aspect, the muscle-specific control element of the rAAV vector is truncated MCK (tMCK). In another aspect, the promoter and/or the muscle-specific control element of the rAAV vector is an MHCK7 promoter. In a further aspect, the MHCK promoter comprises, or consists essentially of, or yet further consists of the nucleotide sequence set forth in SEQ ID NO: 3 or an equivalent thereof and provides promoter function. In one embodiment, the genome of the rAAV vector disclosed herein comprises, or consists essentially of, or yet further consists of an intron comprising the nucleotide sequence set forth in SEQ ID NO: 5.

In some embodiments, the polynucleotide sequence encoding γ-sarcoglycan consists the nucleotide sequence set forth in SEQ ID NO: 1 or a polynucleotide sequence encoding γ-sarcoglycan that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to SEQ ID NO: 1 and retains γ-sarcoglycan activity.

In another aspect, a recombinant AAV vector described herein comprises, or consists essentially of a polynucleotide sequence encoding γ-sarcoglycan that is at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98% or 99% sequence identity to the amino acid sequence of human γ-sarcoglycan, which is set forth in SEQ ID NO: 2 (see Table 1), and the protein retains γ-sarcoglycan activity.

TABLE 1
Non-Limiting Examples of Protein and
Nucleotide Sequences

| | | SEQ |
|---|---|---|
| Sequence | | ID |
| description | Sequence | NO |
| Human γ- | ATGGTGAGGGAGCAGTACACCACAGCAACCGAGGG | 1 |
| sarcoglycan | AATCTGCATCGAGAGGCCAGAGAACCAGTACGTGT | |
| nucleotide | ATAAGATCGGCATCTACGGCTGGCGGAAGAGATGT | |
| sequence | CTGTATCTGTTCGTGCTGCTGCTGCTGATCATCCT | |
| (codon- | GGTGGTGAATCTGGCCCTGACCATCTGGATCCTGA | |
| optimized) | AAGTGATGTGGTTTTCCCCAGCAGGAATGGGACAC | |
| | CTGTGCGTGACAAAGGACGGACTGCGGCTGGAGGG | |
| | AGAGTCTGAGTTCCTGTTTCCCCTGTATGCCAAGG | |
| | AGATCCACAGCAGAGTGGATAGCTCCCTGCTGCTG | |
| | CAGTCCACCCAGAACGTGACAGTGAACGCAAGGAA | |
| | TAGCGAGGGAGAGGTGACCGGCAGACTGAAGGTCG | |
| | GCCCCAAGATGGTGGAGGTGCAGAATCAGCAGTTC | |
| | CAGATCAACTCCAATGACGGCAAGCCTCTGTTTAC | |
| | AGTGGATGAGAAGGAGGTGGTGGTGGGCACCGACA | |
| | AGCTGAGGGTGACAGGACCTGAGGGCGCCCTGTTC | |
| | GAGCACTCTGTGGAGACCCCACTGGTGCGCGCAGA | |
| | CCCTTTTCAGGATCTGAGGCTGGAGAGCCCAACAC | |
| | GCAGCCTGTCCATGGACGCACCCAGAGGCGTGCAC | |
| | ATCCAGGCACACGCAGGCAAGATCGAGGCCCTGAG | |
| | CCAGATGGATATCCTGTTCCACTCTAGCGACGGCA | |
| | TGCTGGTGCTGGATGCCGAGACCGTGTGCCTGCCT | |
| | AAGCTGGTGCAGGGCACATGGGGCCCATCTGGCTC | |
| | CTCTCAGAGCCTGTACGAGATCTGCGTGTGCCCAG | |
| | ATGGCAAGCTGTATCTGTCCGTGGCCGGCGTGTCT | |
| | ACCACATGCCAGGAGCACAACCACATCTGTCTGTG | |
| | A | |
| Human γ- | MVREQYTTATEGICIERPENQYVYKIGIYGWRKRC | 2 |
| sarcoglycan, | LYLFVLLLLIILVVNLALTIWILKVMWFSPAGMGH | |
| amino acid | LCVTKDGLRLEGESEFLFPLYAKEIHSRVDSSLLL | |
| sequence | QSTQNVTVNARNSEGEVTGRLKVGPKMVEVQNQQF | |
| | QINSNDGKPLFTVDEKEVVVGTDKLRVTGPEGALF | |
| | EHSVETPLVRADPFQDLRLESPTRSLSMDAPRGVH | |
| | IQAHAGKIEALSQMDILFHSSDGMLVLDAETVCLP | |
| | KLVQGTWGPSGSSQSLYEICVCPDGKLYLSVAGVS | |
| | TTCQEHNHICL | |
| 5′ITR-MHCK7- | CTGCGCGCTCGCTCGCTCACTGAGGCCGCCCGGGC | 3 |
| Chimeric | AAAGCCCGGGCGTCGGGCGACCTTTGGTCGCCCGG | |
| Intron- | CCTCAGTGAGCGAGCGAGCGCGCAGAGAGGGAGTG | |
| hSGCG-PolyA- | GGGTTAACCAATTGGCGCGGCCGCAAGCTTGCATG | |
| 3′ITR (full | TCTAAGCTAGACCCTTCAGATTAAAAATAACTGAG | |
| sequence | GTAAGGGCCTGGGTAGGGGAGGTGGTGTGAGACGC | |
| between | TCCTGTCTCTCCTCTATCTGCCCATCGGCCCTTTG | |
| ITRs) | GGGAGGAGGAATGTGCCCAAGGACTAAAAAAAGGC | |
| | CATGGAGCCAGAGGGGCGAGGGCAACAGACCTTTC | |
| | ATGGGCAAACCTTGGGGCCCTGCTGTCTAGCATGC | |
| | CCCACTACGGGTCTAGGCTGCCCATGTAAGGAGGC | |
| | AAGGCCTGGGGACACCCGAGATGCCTGGTTATAAT | |
| | TAACCCAGACATGTGGCTGCCCCCCCCCCCCCAAC | |
| | ACCTGCTGCCTCTAAAAATAACCCTGTCCCTGGTG | |
| | GATCCCCTGCATGCGAAGATCTTCGAACAAGGCTG | |
| | TGGGGGACTGAGGGCAGGCTGTAACAGGCTTGGGG | |
| | GCCAGGGCTTATACGTGCCTGGGACTCCCAAAGTA | |
| | TTACTGTTCCATGTTCCCGGCGAAGGGCCAGCTGT | |
| | CCCCCGCCAGCTAGACTCAGCACTTAGTTTAGGAA | |
| | CCAGTGAGCAAGTCAGCCCTTGGGGCAGCCCATAC | |
| | AAGGCCATGGGGCTGGGCAAGCTGCACGCCTGGGT | |
| | CCGGGGTGGGCACGGTGCCCGGGCAACGAGCTGAA | |
| | AGCTCATCTGCTCTCAGGGGCCCCTCCCTGGGGAC | |
| | AGCCCCTCCTGGCTAGTCACACCCTGTAGGCTCCT | |
| | CTATATAACCCAGGGGCACAGGGGCTGCCCTCATT | |
| | CTACCACCACCTCCACAGCACAGACAGACACTCAG | |
| | GAGCAGCCAGCGGCGCGCCCAGGTAAGTTTAGTCT | |
| | TTTTGTCTTTTATTTCAGGTCCCGGATCCGGTGGT | |
| | GGTGCAAATCAAAGAACTGCTCCTCAGTGGATGTT | |
| | GCCTTTACTTCTAGGCCTGTACGGAAGTGTTACTT | |
| | CTGCTCTAAAAGCTGCGGAATTGTACCCGGTACCA | |
| | CCATGGTGAGGGAGCAGTACACCACAGCAACCGAG | |
| | GGAATCTGCATCGAGAGGCCAGAGAACCAGTACGT | |
| | GTATAAGATCGGCATCTACGGCTGGCGGAAGAGAT | |
| | GTCTGTATCTGTTCGTGCTGCTGCTGCTGATCATC | |
| | CTGGTGGTGAATCTGGCCCTGACCATCTGGATCCT | |
| | GAAAGTGATGTGGTTTTCCCCAGCAGGAATGGGAC | |
| | ACCTGTGCGTGACAAAGGACGGACTGCGGCTGGAG | |
| | GGAGAGTCTGAGTTCCTGTTTCCCCTGTATGCCAA | |
| | GGAGATCCACAGCAGAGTGGATAGCTCCCTGCTGC | |
| | TGCAGTCCACCCAGAACGTGACAGTGAACGCAAGG | |
| | AATAGCGAGGGAGAGGTGACCGGCAGACTGAAGGT | |
| | CGGCCCCAAGATGGTGGAGGTGCAGAATCAGCAGT | |
| | TCCAGATCAACTCCAATGACGGCAAGCCTCTGTTT | |
| | ACAGTGGATGAGAAGGAGGTGGTGGTGGGCACCGA | |
| | CAAGCTGAGGGTGACAGGACCTGAGGGCGCCCTGT | |
| | TCGAGCACTCTGTGGAGACCCCACTGGTGCGCGCA | |
| | GACCCTTTTCAGGATCTGAGGCTGGAGAGCCCAAC | |
| | ACGCAGCCTGTCCATGGACGCACCCAGAGGCGTGC | |
| | ACATCCAGGCACACGCAGGCAAGATCGAGGCCCTG | |
| | AGCCAGATGGATATCCTGTTCCACTCTAGCGACGG | |
| | CATGCTGGTGCTGGATGCCGAGACCGTGTGCCTGC | |
| | CTAAGCTGGTGCAGGGCACATGGGGCCCATCTGGC | |
| | TCCTCTCAGAGCCTGTACGAGATCTGCGTGTGCCC | |
| | AGATGGCAAGCTGTATCTGTCCGTGGCCGGCGTGT | |
| | CTACCACATGCCAGGAGCACAACCACATCTGTCTG | |
| | TGACTCGAGGGCCGCAATAAAAGATCTTTATTTTC | |
| | ATTAGATCTGTGTGTTGGTTTTTTGTGTGTCCTGC | |
| | AGGGGCGCGCCTAATCTAGAGCATGGCTACGTAGA | |
| | TAAGTAGCATGGCGGGTTAATCATTAACTACAAGG | |
| | AACCCCTAGTGATGGAGTTGGCCACTCCCTCTCTG | |
| | CGCGCTCGCTCGCTCACTGAGGCCGGGCGACCAAA | |
| | GGTCGCCCGACGCCCGGGCTTTGCCCGGGCGGCCT | |
| | CAGTGAGCGAGCGAGCGCGC | |
| MHCK7 | AAGCTTGCATGTCTAAGCTAGACCCTTCAGATTAA | 4 |
| Promoter | AAATAACTGAGGTAAGGGCCTGGGTAGGGGAGGTG | |
| | GTGTGAGACGCTCCTGTCTCTCCTCTATCTGCCCA | |
| | TCGGCCCTTTGGGGAGGAGGAATGTGCCCAAGGAC | |
| | TAAAAAAAGGCCATGGAGCCAGAGGGGCGAGGGCA | |
| | ACAGACCTTTCATGGGCAAACCTTGGGGCCCTGCT | |
| | GTCTAGCATGCCCCACTACGGGTCTAGGCTGCCCA | |
| | TGTAAGGAGGCAAGGCCTGGGGACACCCGAGATGC | |
| | CTGGTTATAATTAACCCAGACATGTGGCTGCCCCC | |
| | CCCCCCCCAACACCTGCTGCCTCTAAAAATAACCC | |
| | TGTCCCTGGTGGATCCCCTGCATGCGAAGATCTTC | |
| | GAACAAGGCTGTGGGGGACTGAGGGCAGGCTGTAA | |
| | CAGGCTTGGGGGCCAGGGCTTATACGTGCCTGGGA | |
| | CTCCCAAAGTATTACTGTTCCATGTTCCCGGCGAA | |
| | GGGCCAGCTGTCCCCCGCCAGCTAGACTCAGCACT | |
| | TAGTTTAGGAACCAGTGAGCAAGTCAGCCCTTGGG | |
| | GCAGCCCATACAAGGCCATGGGGCTGGGCAAGCTG | |
| | CACGCCTGGGTCCGGGGTGGGCACGGTGCCCGGGC | |
| | AACGAGCTGAAAGCTCATCTGCTCTCAGGGGCCCC | |
| | TCCCTGGGGACAGCCCCTCCTGGCTAGTCACACCC | |
| | TGTAGGCTCCTCTATATAACCCAGGGGCACAGGGG | |
| | CTGCCCTCATTCTACCACCACCTCCACAGCACAGA | |
| | CAGACACTCAGGAGCAGCCAGC | |
| Chimeric | AGGTAAGTTTAGTCTTTTTGTCTTTTATTTCAGGT | 5 |
| Intron | CCCGGATCCGGTGGTGGTGCAAATCAAAGAACTGC | |
| | TCCTCAGTGGATGTTGCCTTTACTTCTAGGCCTGT | |
| | ACGGAAGTGTTACTTCTGCTCTAAAAGCTGCGGAA | |
| | TTGTACCC | |
| PolyA | GGCCGCAATAAAAGATCTTTATTTTCATTAGATCT | 6 |
| | GTGTGTTGGTTTTTTGTG | |
| 5′ ITR | CTGCGCGCTCGCTCGCTCACTGAGGCCGCCCGGGC | 7 |
| | AAAGCCCGGGCGTCGGGCGACCTTTGGTCGCCCGG | |
| | CCTCAGTGAGCGAGCGAGCGCGCAGAGAGGGAGTG | |
| | GGGTT | |
| 3′ ITR | CCACTCCCTCTCTGCGCGCTCGCTCGCTCACTGAG | 8 |
| | GCCGGGCGACCAAAGGTCGCCCGACGCCCGGGCTT | |
| | TGCCCGGGCGGCCTCAGTGAGCGAGCGAGCGCGC | |
| Human γ- | ATGGTGCGTGAGCAGTACACTACAGCCACAGAAGG | 9 |
| sarcoglycan | CATCTGCATAGAGAGGCCAGAGAATCAGTATGTCT | |
| nucleotide | ACAAAATTGGCATTTATGGCTGGAGAAAGCGCTGT | |
| sequence | CTCTACTTGTTTGTTCTTCTTTTACTCATCATCCT | |
| (wild-type), | CGTTGTGAATTTAGCTCTTACAATTTGGATTCTTA | |
| GenBank | AAGTGATGTGGTTTTCTCCAGCAGGAATGGGCCAC | |
| U34976.1 | TTGTGTGTAACAAAAGATGGACTGCGCTTGGAAGG | |
| | GGAATCAGAATTTTTATTCCCATTGTATGCCAAAG | |
| | AAATACACTCCAGAGTGGACTCATCTCTGCTGCTA | |
| | CAATCAACCCAGAATGTGACTGTAAATGCGCGCAA | |
| | CTCAGAAGGGGAGGTCACAGGCAGGTTAAAAGTCG | |
| | GTCCCAAAATGGTAGAAGTCCAGAATCAACAGTTT | |
| | CAGATCAACTCCAACGACGGCAAGCCACTATTTAC | |
| | TGTAGATGAGAAGGAAGTTGTGGTTGGTACAGATA | |
| | AACTTCGAGTAACTGGGCCTGAAGGGGCTCTTTTT | |
| | GAACATTCAGTGGAGACACCCCTTGTCAGAGCCGA | |
| | CCCGTTTCAAGACCTTAGATTAGAATCCCCCACTC | |
| | GGAGTCTAAGCATGGATGCCCCAAGGGGTGTGCAT | |
| | ATTCAAGCTCACGCTGGGAAAATTGAGGCGCTTTC | |
| | TCAAATGGATATTCTTTTTCATAGTAGTGATGGAA | |
| | TGCTTGTGCTTGATGCTGAAACTGTGTGCTTACCC | |
| | AAGCTGGTGCAGGGGACGTGGGGTCCCTCTGGCAG | |
| | CTCACAGAGCCTCTACGAAATCTGTGTGTGTCCAG | |
| | ATGGGAAGCTGTACCTGTCTGTGGCCGGTGTGAGC | |
| | ACCACGTGCCAGGAGCACAGCCACATCTGCCTCTG | |
| | A | |
| AAV rh.74 | MAAGGGAPMADNNEGADGVGSSSGNWHCDSTWLGD | 10 |
| capsid | RVITTSTRTWALPTYNNHLYKQISNGTSGGSTNDN | |
| amino acid | TYFGYSTPWGYFDFNRFHCHFSPRDWQRLINNNWG | |
| sequence | FRPKRLNFKLFNIQVKEVTQNEGTKTIANNLTSTI | |
| | QVFTDSEYQLPYVLGSAHQGCLPPFPADVFMIPQY | |
| | GYLTLNNGSQAVGRSSFYCLEYFPSQMLRTGNNFE | |
| | FSYNFEDVPFHSSYAHSQSLDRLMNPLIDQYLYYL | |
| | SRTQSTGGTAGTQQLLFSQAGPNNMSAQAKNWLPG | |
| | PCYRQQRVSTTLSQNNNSNFAWTGATKYHLNGRDS | |
| | LVNPGVAMATHKDDEERFFPSSGVLMFGKQGAGKD | |
| | NVDYSSVMLTSEEEIKTTNPVATEQYGVVADNLQQ | |
| | QNAAPIVGAVNSQGALPGMVWQNRDVYLQGPIWAK | |
| | IPHTDGNFHPSPLMGGFGLKHPPPQILIKNTPVPA | |
| | DPPTTFNQAKLASFITQYSTGQVSVEIEWELQKEN | |
| | SKRWNPEIQYTSNYYKSTNVDFAVNTEGTYSEPRP | |
| | IGTRYLTRNL | |
| rAAV vector | ATGGCTGCCGATGGTTATCTTCCAGATTGGCTCGA | 11 |
| poly- | GGACAACCTCTCTGAGGGCATTCGCGAGTGGTGGG | |
| nucleotide | ACCTGAAACCTGGAGCCCCGAAACCCAAAGCCAAC | |
| sequence | CAGCAAAAGCAGGACAACGGCCGGGGTCTGGTGCT | |
| | TCCTGGCTACAAGTACCTCGGACCCTTCAACGGAC | |
| | TCGACAAGGGGGAGCCCGTCAACGCGGCGGACGCA | |
| | GCGGCCCTCGAGCACGACAAGGCCTACGACCAGCA | |
| | GCTCCAAGCGGGTGACAATCCGTACCTGCGGTATA | |
| | ATCACGCCGACGCCGAGTTTCAGGAGCGTCTGCAA | |
| | GAAGATACGTCTTTTGGGGGCAACCTCGGGCGCGC | |
| | AGTCTTCCAGGCCAAAAAGCGGGTTCTCGAACCTC | |
| | TGGGCCTGGTTGAATCGCCGGTTAAGACGGCTCCT | |
| | GGAAAGAAGAGACCGGTAGAGCCATCACCCCAGCG | |
| | CTCTCCAGACTCCTCTACGGGCATCGGCAAGAAAG | |
| | GCCAGCAGCCCGCAAAAAAGAGACTCAATTTTGGG | |
| | CAGACTGGCGACTCAGAGTCAGTCCCCGACCCTCA | |
| | ACCAATCGGAGAACCACCAGCAGGCCCCTCTGGTC | |
| | TGGGATCTGGTACAATGGCTGCAGGCGGTGGCGCT | |
| | CCAATGGCAGACAATAACGAAGGCGCCGACGGAGT | |
| | GGGTAGTTCCTCAGGAAATTGGCATTGCGATTCCA | |
| | CATGGCTGGGCGACAGAGTCATCACCACCAGCACC | |
| | CGCACCTGGGCCCTGCCCACCTACAACAACCACCT | |
| | CTACAAGCAAATCTCCAACGGGACCTCGGGAGGAA | |
| | GCACCAACGACAACACCTACTTCGGCTACAGCACC | |
| | CCCTGGGGGTATTTTGACTTCAACAGATTCCACTG | |
| | CCACTTTTCACCACGTGACTGGCAGCGACTCATCA | |
| | ACAACAACTGGGGATTCCGGCCCAAGAGGCTCAAC | |
| | TTCAAGCTCTTCAACATCCAAGTCAAGGAGGTCAC | |
| | GCAGAATGAAGGCACCAAGACCATCGCCAATAACC | |
| | TTACCAGCACGATTCAGGTCTTTACGGACTCGGAA | |
| | TACCAGCTCCCGTACGTGCTCGGCTCGGCGCACCA | |
| | GGGCTGCCTGCCTCCGTTCCCGGCGGACGTCTTCA | |
| | TGATTCCTCAGTACGGGTACCTGACTCTGAACAAT | |
| | GGCAGTCAGGCTGTGGGCCGGTCGTCCTTCTACTG | |
| | CCTGGAGTACTTTCCTTCTCAAATGCTGAGAACGG | |
| | GCAACAACTTTGAATTCAGCTACAACTTCGAGGAC | |
| | GTGCCCTTCCACAGCAGCTACGCGCACAGCCAGAG | |
| | CCTGGACCGGCTGATGAACCCTCTCATCGACCAGT | |
| | ACTTGTACTACCTGTCCCGGACTCAAAGCACGGGC | |
| | GGTACTGCAGGAACTCAGCAGTTGCTATTTTCTCA | |
| | GGCCGGGCCTAACAACATGTCGGCTCAGGCCAAGA | |
| | ACTGGCTACCCGGTCCCTGCTACCGGCAGCAACGC | |
| | GTCTCCACGACACTGTCGCAGAACAACAACAGCAA | |
| | CTTTGCCTGGACGGGTGCCACCAAGTATCATCTGA | |
| | ATGGCAGAGACTCTCTGGTGAATCCTGGCGTTGCC | |
| | ATGGCTACCCACAAGGACGACGAAGAGCGATTTTT | |
| | TCCATCCAGCGGAGTCTTAATGTTTGGGAAACAGG | |
| | GAGCTGGAAAAGACAACGTGGACTATAGCAGCGTG | |
| | ATGCTAACCAGCGAGGAAGAAATAAAGACCACCAA | |
| | CCCAGTGGCCACAGAACAGTACGGCGTGGTGGCCG | |
| | ATAACCTGCAACAGCAAAACGCCGCTCCTATTGTA | |
| | GGGGCCGTCAATAGTCAAGGAGCCTTACCTGGCAT | |
| | GGTGTGGCAGAACCGGGACGTGTACCTGCAGGGTC | |
| | CCATCTGGGCCAAGATTCCTCATACGGACGGCAAC | |
| | TTTCATCCCTCGCCGCTGATGGGAGGCTTTGGACT | |
| | GAAGCATCCGCCTCCTCAGATCCTGATTAAAAACA | |
| | CACCTGTTCCCGCGGATCCTCCGACCACCTTCAAT | |
| | CAGGCCAAGCTGGCTTCTTTCATCACGCAGTACAG | |
| | TACCGGCCAGGTCAGCGTGGAGATCGAGTGGGAGC | |
| | TGCAGAAGGAGAACAGCAAACGCTGGAACCCAGAG | |
| | ATTCAGTACACTTCCAACTACTACAAATCTACAAA | |
| | TGTGGACTTTGCTGTCAATACTGAGGGTACTTATT | |
| | CCGAGCCTCGCCCCATTGGCACCCGTTACCTCACC | |
| | CGTAATCTGTAA | |

In another aspect, described herein is a recombinant AAV vector comprising a polynucleotide sequence encoding functional γ-sarcoglycan that comprises, or consists essentially of, or yet further consists of a nucleotide sequence that hybridizes under stringent conditions to the nucleic acid sequence of SEQ ID NO: 1, or a complement thereof. Functional γ-sarcoglycan intends a γ-sarcoglycan polypeptide that retains γ-sarcoglycan activity. γ-sarcoglycan activity is critical for muscle function. γ-sarcoglycan is one of several sarcolemmal transmembrane glycoproteins that interact with dystrophin and forms the dystrophin-glycoprotein complex, which spans the sarcolemma and is comprised of dystrophin, syntrophin, α-dystroglycans and β-dystroglycans, and sarcoglycans including γ-sarcoglycan. The dystrophin-glycoprotein complex provides a structural link between the subsarcolemmal cytoskeleton and the extracellular matrix of muscle cells. Non-limiting example of muscle cells include cardiac, diaphragm, leg, pelvic girdle, shoulder and arm muscle cells. Further non-limiting examples of γ-sarcoglycan activity and consequences of γ-sarcoglycanopathy are described in Blake et al. (2002) Physiol Rev.; 82 (2): 291-329 and Tarakci et al. (2016) Front Biosci (Landmark Ed); 21:744-56.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015 M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodS04, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

In another aspect, the recombinant AAV vectors described herein may comprise, or consist essentially of, or yet further consist of a polynucleotide sequence encoding γ-sarcoglycan that is operably linked to a promoter and/or a muscle-specific control element. For example the muscle-specific control element is human skeletal actin gene element, cardiac actin gene element, myocyte-specific enhancer binding factor MEF, muscle creatine kinase (MCK), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, the slow-twitch troponin I gene element, hypozia-inducible nuclear factors, steroid-inducible element or glucocorticoid response element (GRE). In one embodiment, a rAAV vector comprises the MHCK7 promoter (SEQ ID NO: 4).

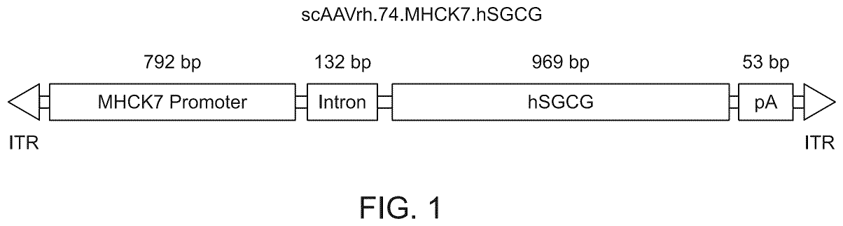

An exemplary rAAV vector described herein is pAAV.MHCK7.hSCGC, which comprises the nucleotide sequence of SEQ ID NO: 3; wherein the MCHK7 promoter spans nucleotides 136-927 (SEQ ID NO: 4), an intron spans nucleotides 937-1084 (SEQ ID NO: 5), the γ-sarcoglycan sequence spans nucleotides 1094-1969 (SEQ ID NO: 1) and the polyA spans nucleotides 1976-2028 (SEQ ID NO: 6). See FIG. 1. In some cases, the only viral sequences included in a rAAV vector are the inverted terminal repeats, which are required for viral DNA replication and packaging. In some cases, the intron (SEQ ID NO: 5), spanning nucleotides 7-116, and the 5′ UTR (SEQ ID NO: 7), spanning nucleotides 2128-2231, are derived from plasmid pCMVβ (Clontech). In certain cases, the 3′ UTR comprises the sequence set forth in SEQ ID NO: 8. In certain cases, pAAV.MHCK7.hSCGC is packaged in an AAV rh.74 capsid.

DNA plasmids of the disclosure comprise rAAV genomes. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 and AAV rh.74. In some embodiments, a rAAV vector comprises the inverted terminal repeat (ITR) sequences of AAV2. Production of pseudotyped rAAV is disclosed in, for example, WO 01/83692 which is incorporated by reference herein in its entirety. In certain aspects, a rAAV vector comprises the inverted ITR sequences of AAV2 and is encapsidated in a capsid of AAV rh.74. In certain cases, the genome of the rAAV vector comprises the polynucleotide sequence set forth in SEQ ID NO: 11. In certain cases, the AAV rh.74 capsid comprises the amino acid sequence set forth in SEQ ID NO: 10. In some embodiments, the rAAV vector comprises a polynucleotide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 11 and encodes the capsid proteins VP1, VP2, and VP3 of the rAAV. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAV rh.74 VP3 which is set forth in SEQ ID NO: 10.

A method of generating a packaging cell line is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells. In certain cases, the genome of the rAAV vector comprises the polynucleotide sequence set forth in SEQ ID NO: 11. In certain cases, the AAV rh.74 capsid comprises the amino acid sequence set forth in SEQ ID NO: 10. In some embodiments, the rAAV vector comprises a polynucleotide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to the nucleotide sequence set forth in SEQ ID NO: 11 and encodes the capsid proteins VP1, VP2, and VP3 of the rAAV. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAV rh.74 VP3 which is set forth in SEQ ID NO: 10.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the disclosure comprise a rAAV genome. Embodiments include, but are not limited to, the rAAV named pAAV.MHCK7.hSCGC which comprises the polynucleotide sequence set forth in SEQ ID NO: 3.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

In another embodiment, the disclosure contemplates compositions comprising, or consisting essentially of rAAV of the present disclosure. Compositions described herein comprise, or consist essentially of rAAV in a pharmaceutically acceptable carrier. In one particular embodiment, the composition of this disclosure comprise, or consist essentially of of Lactated Ringer's Solution (LRS). The compositions may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-formig counterions such as sodium; and/or nonionic surfactants such as Tween, pluronics or polyethylene glycol (PEG). The compositions disclosed can be used for one or more of for one or more of treating γ-sarcoglycanopathy; increasing muscular force, muscle endurance, and/or muscle mass; reducing fibrosis; reducing contraction-induced injury; decreasing fatty infiltration; and/or decreasing central nucleation in a subject in need thereof, and/or treating muscular dystrophy reducing degenerating fibers or necrotic fibers; reducing inflammation; elevating creatine kinase levels; treating myofiber atrophy and hypertrophy, and/or decreasing dystrophic calcification in a subject suffering from muscular dystrophy.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg).

Methods of transducing a target cell with rAAV, in vivo or in vitro, are contemplated by the disclosure. The term “transduction” is used to refer to the administration/delivery of a polynucleotide of interest (e.g., a polynucleotide sequence encoding γ-sarcoglycan) to a recipient cell either in vivo or in vitro, via a replication-deficient rAAV described resulting in expression of γ-sarcoglycan by the recipient cell.

In one aspect provided herein are a methods for one or more of treating γ-sarcoglycanopathy; increasing muscular force, muscle endurance, and/or muscle mass; reducing fibrosis; reducing contraction-induced injury; decreasing fatty infiltration; and/or decreasing central nucleation in a subject in need thereof, and/or treating muscular dystrophy reducing degenerating fibers or necrotic fibers; reducing inflammation; elevating creatine kinase levels; treating myofiber atrophy and hypertrophy, and/or decreasing dystrophic calcification in a subject suffering from muscular dystrophy, comprising, or consisting essentially of, or yet further consisting of administering to the subject a therapeutically effective amount of a recombinant adeno-associated virus (AAV) vector, wherein the rAAV vector comprises, or consists essentially of, or yet further consists of a gene expression cassette comprising, or consisting essentially of, or yet further consisting of a polynucleotide sequence encoding γ-sarcoglycan under the transcriptional control of a promoter, said cassette flanked by one or more AAV inverted terminal repeats. In some embodiments, said promoter is a muscle-specific control element. In one embodiment, the methods disclosed herein increase muscular force, muscle endurance, and/or muscle mass of one or more muscles of the subject. Non-limiting examples of muscles include heart, diaphragm, upper legs, lower legs, pelvic girdle shoulder, and arm muscles. In one specific embodiment, the muscular force, muscle endurance, and/or muscle mass is increased at least about 5%, at least about 10%, at least about 15%, at least about 20%, at least about 50%, or at least about 80% compared to an untreated control subject.

In one particular aspect, the subject is suffering from limb-girdle muscular dystrophy. In a further aspect, the subject is suffering from limb-girdle muscular dystrophy is limb-girdle muscular dystrophy type 2C.

The terms “administering” or “administration” in reference to delivering the polynucleotides to a subject include any route of introducing or delivering to a subject the polynucleotides to perform the intended function. Administration can be carried out by any suitable route, including orally, intranasally, parenterally (intravenously, intramuscularly, intraperitoneally, or subcutaneously), intracranially, or topically. Additional routes of administration include intraorbital, infusion, intraarterial, intracapsular, intracardiac, intradermal, intrapulmonary, intraspinal, intrasternal, intrathecal, intrauterine, intravenous, subarachnoid, subcapsular, subcutaneous, transmucosal, or transtracheal. Administration includes self-administration and the administration by another.

In one aspect, the methods disclosed herein comprise, or consist essentially of, or yet further consist of administering a composition comprising, or consisting essentially of, or yet further consisting of the rAAV vector and a pharmaceutically acceptable carrier. In a further aspect, the methods disclosed herein comprise, or consist essentially of, or yet further consist of administering the rAAV vector or the composition comprising, or consisting essentially of, or yet further consisting of the rAAV vector and a pharmaceutically acceptable carrier by intramuscular injection or intravenous injection. In a yet further aspect, the methods disclosed herein comprise, or consist essentially of, or yet further consist of administering the rAAV vector or the composition comprising, or consisting essentially of, or yet further consisting of the rAAV vector and a pharmaceutically acceptable carrier systemically. In one particular aspect, the methods disclosed herein comprise, or consist essentially of, or yet further consist of administering the rAAV vector or the composition comprising, or consisting essentially of, or yet further consisting of the rAAV vector and a pharmaceutically acceptable carrier parentally by injection, infusion, or implantation.

In one aspect, the polynucleotide sequence encoding γ-sarcoglycan of the rAAV vector for use in the methods described herein comprises, or consists essentially of, or yet further consists of the nucleotide sequence set forth in SEQ ID NO: 1. In another aspect, the polynucleotide sequence encoding γ-sarcoglycan of the rAAV vector encodes the amino acid sequence of SEQ ID NO: 2. In yet another aspect, the rAAV vector used in the methods disclosed herein comprises, or consists essentially of, or yet further consists of a self-complementary AAV vector genome. In one particular embodiment, the rAAV vector comprises, or consists essentially of, or yet further consists of a genome lacking AAV rep and cap DNA. In another embodiment, the rAAV vector is of the serotype AAV1, AAV2, AAV4, AAV5, AAV6, AAV7, AAV8, AAV9, AAV10, AAV11, AAV12, AAV13 or AAV rh74. In a further aspect, the rAAV vector is of the serotype AAV rh74 and the rAAV vector comprises, or consists essentially of, or yet further consists of an AAV rh.74 capsid. In a yet further aspect, AAV rh.74 capsid of the rAAV vector comprises, or consists essentially of, or yet further consists of the amino acid sequence set forth in SEQ ID NO: 10 or an equivalent thereof.

The rAAV vector used in the methods disclosed herein may further comprise, or consist essentially of a promoter and/or a muscle-specific control element and wherein the muscle-specific control element is operatively linked to the polynucleotide encoding γ-sarcoglycan. Non-limiting examples some muscle-specific control elements are human skeletal actin gene element, cardiac actin gene element, myocyte-specific enhancer binding factor mef, muscle creatine kinase (MCK), truncated MCK (tMCK), myosin heavy chain (MHC), MHCK7, C5-12, murine creatine kinase enhancer element, skeletal fast-twitch troponin c gene element, slow-twitch cardiac troponin c gene element, the slow-twitch troponin I gene element, hypoxia-inducible nuclear factors, steroid-inducible element, and glucocorticoid response element (gre). In one aspect, the muscle-specific control element of the rAAV vector is truncated MCK (tMCK). In another aspect, the promoter and/or the muscle-specific control element of the rAAV vector is an MHCK7 promoter. In a further aspect, the MHCK promoter comprises, or consists essentially of, or yet further consists of the nucleotide sequence set forth in SEQ ID NO: 3 or an equivalent thereof.

In one embodiment, the genome of the rAAV vector disclosed herein comprises an intron comprising the nucleotide sequence set forth in SEQ ID NO: 5.

The in vivo methods comprise, or consist essentially of, or yet further consist of the step of administering an effective dose, or effective multiple doses, of a composition comprising, or consisting essentially of, or yet further consisting of a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the disclosure is muscular dystrophy, such as limb-girdle muscular dystrophy. In some embodiments, a disease contemplated for prevention or treatment with methods of the disclosure is limb-girdle muscular dystrophy type 2C (LGMD2C).

The term “muscular dystrophy” as used herein refers to a disorder in which strength and muscle bulk gradually decline. Non-limiting examples of muscular dystrophy diseases may include Becker muscular dystrophy, tibial muscular dystrophy, Duchenne muscular dystrophy, Emery-Dreifuss muscular dystrophy, facioscapulohumeral muscular dystrophy, sarcoglycanopathies, congenital muscular dystrophy such as congenital muscular dystrophy due to partial LAMA2 deficiency, merosin-deficient congenital muscular dystrophy, type ID congenital muscular dystrophy, Fukuyama congenital muscular dystrophy, limb-girdle type 1A muscular dystrophy, limb-girdle type 2A muscular dystrophy, limb-girdle type 2B muscular dystrophy, limb-girdle type 2C muscular dystrophy, limb-girdle type 2D muscular dystrophy, limb-girdle type 2E muscular dystrophy, limb-girdle type 2F muscular dystrophy, limb-girdle type 2G muscular dystrophy, limb-girdle type 2H muscular dystrophy, limb-girdle type 21 muscular dystrophy, limb-girdle type 21 muscular dystrophy, limb-girdle type 2J muscular dystrophy, limb-girdle type 2K muscular dystrophy, limb-girdle type IC muscular dystrophy, rigid spine muscular dystrophy with epidermolysis bullosa simplex, oculopharyngeal muscular dystrophy, Ullrich congenital muscular dystrophy, and Ullrich scleroatonic muscular dystrophy. In some embodiments, the subject is suffering from limb-girdle muscular dystrophy. In some embodiments, the subject is suffering from limb-girdle muscular dystrophy type 2C (LGMD2C).

There are at least nineteen forms of LGMD, and the forms are classified by their associated genetic defects.

| Type | Pattern of Inheritance | Gene or Chromosome |
|---|---|---|
| LGMD1A | Autosomal dominant | Myotilin gene |
| LGMD1B | Autosomal dominant | Lamin A/C gene |
| LGMD1C | Autosomal dominant | Caveolin gene |
| LGMD1D | Autosomal dominant | Chromosome 7 |
| LGMD1E | Autosomal dominant | Chromosome 6 |
| LGMD1F | Autosomal dominant | Chromosome 7 |
| LGMD1G | Autosomal dominant | Chromosome 4 |
| LGMD2A | Autosomal recessive | Calpain-3 gene |
| LGMD2B | Autosomal recessive | Dysferlin gene |
| LGMD2C | Autosomal recessive | Gamma-sarcoglycan gene |
| LGMD2D | Autosomal recessive | Alpha-sarcoglycan gene |
| LGMD2E | Autosomal recessive | Beta-sarcoglycan gene |
| LGMD2F | Autosomal recessive | Delta-sarcoglycan gene |
| LGMD2G | Autosomal recessive | Telethonin gene |
| LGMD2H | Autosomal recessive | TRIM32 |
| LGMD2I | Autosomal recessive | FKRP gene |
| LGMD2J | Autosomal recessive | Titin gene |
| LGMD2K | Autosomal recessive | POMT1 gene |
| LGMD2L | Autosomal recessive | ANO5 gene |

In some aspects, the disclosure relates to a method of treating muscular dystrophy (e.g., LGMD2C) in a subject, the method comprising, or consisting essentially of, or yet further consisting of administering to the subject a therapeutically effective amount of a rAAV vector encoding γ-sarcoglycan as described herein or a composition comprising, or consisting essentially of such a rAAV vector.

In some embodiments, the disclosure provides a method of increasing muscular force, muscle endurance and/or muscle mass in a subject suffering from muscular dystrophy (e.g., LGMD2C), the method comprising, or consisting essentially of, or yet further consisting of administering to the subject a therapeutically effective amount of a rAAV vector encoding γ-sarcoglycan as described herein or a composition comprising, or consisting essentially of such a rAAV vector.

In certain aspects, the disclosure encompasses a method of reducing contraction-induced injury in a subject suffering from muscular dystrophy (e.g., LGMD2C), the method comprising, or consisting essentially of, or yet further consisting of administering to the subject a therapeutically effective amount of a rAAV vector encoding γ-sarcoglycan as described herein or a composition comprising, or consisting essentially of such a rAAV vector.

In certain aspects, the disclosure encompasses a method of treating γ-sarcoglycanopathy in a subject, the method comprising, or consisting essentially of, or yet further consisting of administering to the subject a therapeutically effective amount of a rAAV vector encoding γ-sarcoglycan as described herein or a composition comprising, or consisting essentially of such a rAAV vector.

The disclosure also encompasses a method of reducing fibrosis in a subject suffering from muscular dystrophy (e.g., LGMD2C), the method comprising, or consisting essentially of, or yet further consisting of administering to the subject a therapeutically effective amount of a rAAV vector encoding γ-sarcoglycan as described herein or a composition comprising, or consisting essentially of such a rAAV vector. The term “fibrosis” as used herein refers to the excessive or unregulated deposition of extracellular matrix (ECM) components and abnormal repair processes in tissues upon injury including skeletal muscle, cardiac muscle, liver, lung, kidney, and pancreas. The ECM components that are deposited include collagen, e.g., collagen 1, collagen 2 or collagen 3, and fibronectin.

In certain embodiments, a subject treated by the methods described herein may be a mammal. In some cases, a subject is a human, a non-human primate, a pig, a horse, a cow, a dog, a cat, a rabbit, a mouse or a rat. A subject may be a human female or a human male. In some cases, the subject is a human subject between the ages of 1-7, 7-15, 16-25, 26-50, 50-70, or greater than 70 years of age. Other age ranges are contemplated and include without limitation, 5-10, 10-15, 15-20, 20-25, 25-30, 30-40, 40-50, 60-70, or greater than 70 years of age, as well as any range comprised by the foregoing.

As used herein, the term “patient in need” or “subject in need” refers to a patient or subject at risk of, or suffering from, a disease, disorder or condition that is amenable to treatment or amelioration with a rAAV comprising a nucleic acid sequence encoding γ-sarcoglycan or a composition comprising, or consisting essentially of such a rAAV provided herein. A patient or subject in need may, for instance, be a patient or subject diagnosed with a disease associated with the malfunction of γ-sarcoglycan, such as LGMD2C. A subject may have a mutation or a malfunction in a γ-sarcoglycan gene or protein. “Subject” and “patient” are used interchangeably herein.

Combination therapies comprising, or consisting essentially of, or yet further consisting of one or more of the compositions disclosed herein and a corticosteroid are also contemplated by the disclosure. Combination as used herein includes simultaneous treatment or sequential treatment. Combinations of methods of the disclosure with standard medical treatments (e.g., corticosteroids) are specifically contemplated, as are combinations with novel therapies. In some embodiments, a subject may be treated with a steroid (e.g., prednisone, prednisolone, deflazacort) to prevent or to reduce an immune response to administration of a rAAV described herein. In certain cases, a subject may receive apheresis or another immune modulator if the subject expresses antibodies to the rAAV described herein.

A therapeutically effective amount of the rAAV vector is in some embodiments a dose of rAAV ranging in one or more administrations in ranges from about 1e13 vg/kg to about 5e14 vg/kg, or about 1e13 vg/kg to about 2e13 vg/kg, or about 1e13 vg/kg to about 3e13 vg/kg, or about 1e13 vg/kg to about 4e13 vg/kg, or about 1e13 vg/kg to about 5e13 vg/kg, or about 1e13 vg/kg to about 6e13 vg/kg, or about 1e13 vg/kg to about 7e13 vg/kg, or about 1e13 vg/kg to about 8e13 vg/kg, or about 1e13 vg/kg to about 9e13 vg/kg, or about 1e13 vg/kg to about 1e14 vg/kg, or about 1e13 vg/kg to about 2e14 vg/kg, or lei 3 vg/kg to about 3e14 vg/kg, or about 1×13 to about 4e14 vg/kg, or about 3e13 vg/kg to about 4e13 vg/kg, or about 3e13 vg/kg to about 5e13 vg/kg, or about 3e13 vg/kg to about 6e13 vg/kg, or about 3e13 vg/kg to about 7e13 vg/kg, or about 3e13 vg/kg to about 8e13 vg/kg, or about 3e13 vg/kg to about 9e13 vg/kg, or about 3e13 vg/kg to about 1e14 vg/kg, or about 3e13 vg/kg to about 2e14 vg/kg, or 3e13 vg/kg to about 3e14 vg/kg, or about 3e13 to about 4e14 vg/kg, or about 3e13 vg/kg to about 5e14 vg/kg, or about 5e13 vg/kg to about 6e13 vg/kg, or about 5e13 vg/kg to about 7e13 vg/kg, or about 5e13 vg/kg to about 8e13 vg/kg, or about 5e13 vg/kg to about 9e13 vg/kg, or about 5e13 vg/kg to about 1e14 vg/kg, or about 5e13 vg/kg to about 2e14 vg/kg, or 5e13 vg/kg to about 3e14 vg/kg, or about 5e13 to about 4e14 vg/kg, or about 5e13 vg/kg to about 5e14 vg/kg, or about 1e14 vg/kg to about 2e14 vg/kg, or 1e14 vg/kg to about 3e14 vg/kg, or about 1e14 to about 4e14 vg/kg, or about 1e14 vg/kg to about 5e14 vg/kg. The disclosure also comprises, or consists essentially of, or yet further consists of compositions comprising, or consisting essentially of, or yet further consisting of these ranges of rAAV vector.

For example, a therapeutically effective amount of rAAV vector is a dose of 1e13 vg/kg, about 2e13 vg/kg, about 3e13 vg/kg, about 4e13 vg/kg, about 5e13 vg/kg, about 6e13 vg/kg, about 7e13 vg/kg, about 8e13 vg/kg, about 9e13 vg/kg, about 1e14 vg/kg, about 2e14 vg/kg, about 3e14 vg/kg, about 4e14 vg/kg and 5e14 vg/kg. The disclosure also comprises, or consists essentially of, or yet further consists of compositions comprising, or consisting essentially of, or yet further consisting of these doses of rAAV vector.

A therapeutic effective amount of rAAV is in some embodiments a dose of rAAV ranging from about 1e14 vg/kg to about 1e15 vg/kg or about 1e15 vg/kg to about 1e16 vg/kg. In some embodiments, the disclosure provides methods of administering an rAAV vector of the disclosure to subject at a dose of about 1e14 vg/kg, about 1.5e14 vg/kg, about 2e14 vg/kg, about 2.5e14 vg/kg, about 3e14 vg/kg, about 3.5e14 vg/kg, about 4e14 vg/kg, about 4.5e14 vg/kg, about 5e14 vg/kg, about 5.5e14 vg/kg, about 6e14 vg/kg, about 6.5e14 vg/kg, about 7e14 vg/kg, about 7.5e14 vg/kg, about 8e14 vg/kg, about 8.5e14 vg/kg, about 9e14 vg/kg, about 9.5e14 vg/kg, about 1e15 vg/kg, about 1.5e15 vg/kg, about 2e15 vg/kg, about 2.5e15 vg/kg, about 3e15 vg/kg, about 3.5e15 vg/kg, about 4e15 vg/kg, about 4.5e15 vg/kg, or about 5e15 vg/kg. In some embodiments, the disclosure provides methods of administering an rAAV vector of the disclosure to subject at a total dose of about 4.0e14 vg/kg, about 4.1e14 vg/kg, about 4.2e14 vg/kg, about 4.3e14 vg/kg, about 4.4e14 vg/kg, about 4.5e14 vg/kg, about 4.6e14 vg/kg, about 4.7e14 vg/kg, about 4.8e14 vg/kg, about 4.9e14 vg/kg, about 5.0e14 vg/kg, about 5.1e14 vg/kg, about 5.2e14 vg/kg, about 5.3e14 vg/kg, about 5.4e14 vg/kg, about 5.5e14 vg/kg, about 5.6e14 vg/kg, about 5.7e14 vg/kg, about 5.8e14 vg/kg, about 5.9e14 vg/kg, or about 6e14 vg/kg.

In various embodiments, the administering step may comprise administering the total dose in 1, 2, 3, 4, 5, 6, 7, 8, 9, 10 or more divided doses. For examples, the total dose may be delivered by injection to multiple sites on the subject or to the subject spaced over several minutes, several hours, or several days.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the infection and/or disease state being treated and the target cells/tissue(s) that are to express the γ-sarcoglycan.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parental administration through injection, infusion or implantation.

In particular, actual administration of rAAV of the present disclosure may be accomplished by using any physical method that will transport the rAAV recombinant vector into the target tissue of an animal. Administration according to the disclosure includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV).

Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the disclosure results in sustained expression of γ-sarcoglycan. The present disclosure thus provides methods of administering/delivering rAAV which express γ-sarcoglycan to a mammalian subject, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one embodiment of the disclosure provides methods of transducing muscle cells and muscle tissues directed by muscle specific control elements, including, but not limited to, those derived from the actin and myosin gene families, such as from the myoD gene family [See Weintraub et ah, Science, 251:761-766 (1991)], the myocyte-specific enhancer binding factor MEF-2 [Cserjesi and Olson, Mol Cell Biol 11:4854-4862 (1991)], control elements derived from the human skeletal actin gene [Muscat et al, Mol Cell Biol, 7:4089-4099 (1987)], the cardiac actin gene, muscle creatine kinase sequence elements [See Johnson et ah, Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et ah, Proc Natl Acad Sci USA, 88:5680-5684 (1991)), steroid-inducible elements and promoters including the glucocorticoid response element (GRE) (See Mader and White, Proc. Natl. Acad. Sci. USA 90:5603-5607 (1993)), and other control elements.

Muscle tissue is an attractive target for in vivo DNA delivery, because it is not a vital organ and is easy to access. The disclosure contemplates sustained expression of a transgene (e.g., γ-sarcoglycan) from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

Thus, also described herein are methods of administering an effective dose (or doses, administered essentially simultaneously or doses given at intervals) of rAAV that encode γ-sarcoglycan to a mammalian subject in need thereof.

Further provided herein are kits comprising, or consisting essentially of, or yet further consisting of, any of one or more of the embodiments disclosed herein and optional instructions for use. The kits can comprise, or consist essentially of, or yet further consist of one or more of the compositions disclosed herein and a corticosteroid or one or more of the combination therapy provided herein and optional instructions for use.

It is to be understood that the present disclosure is not limited to particular aspects described, as such may, of course, vary. It is also to be understood that the terminology used herein is for the purpose of describing particular embodiments only, and is not intended to be limiting, since the scope of the present disclosure will be limited only by the appended claims.

A number of embodiments of the disclosure have been described. Nevertheless, it will be understood that various modifications may be made without departing from the spirit and scope of the disclosure. Accordingly, the following examples are intended to illustrate but not limit the scope of disclosure described in the claims.

It is to be inferred without explicit recitation and unless otherwise intended, that when the present technology relates to a polypeptide, protein, polynucleotide or antibody, an equivalent or a biologically equivalent of such is intended within the scope of the present technology.

Citation of any patent, patent application, publication or any other document is not an admission that any of the foregoing is pertinent prior art, nor does it constitute any admission as to the contents or date of these publications or documents.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation and use of such terms and expressions do not exclude any equivalents of the features shown and described or segments thereof, and various modifications are possible within the scope of the technology claimed.

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%. It should be understood that the terms “a” and “an” as used herein refer to “one or more” of the enumerated components unless otherwise indicated. The use of the alternative (e.g., “or”) should be understood to mean either one, both, or any combination thereof of the alternatives. The term “and/or” should be understood to mean either one, or both of the alternatives. As used herein, the terms “include” and “comprise” are used synonymously.

The section headings used herein are for organizational purposes only and are not to be construed as limiting the subject matter described.

The disclosure is further described in the following Examples, which do not limit the scope of the disclosure described in the claims.

### EXAMPLES

### Example 1: ScAAVrh74.tMCK.hSGCB Construction and Vector Potency

SGCG AAV construct containing a codon-optimized full-length human γ-sarcoglycan (SCGB) cDNA (SEQ ID NO: 1) as shown in FIG. 1 was constructed. The SGCG AAV construct was configured to be packaged using a self-complementary AAV backbone for more efficient transduction efficiency. The SGCG cDNA (969) was configured to be driven by a MHCK7 promoter (792 bp). The intron and 5′ UTR were derived from plasmid pCMVß (Clontech). The SGCG AAV construct had a consensus Kozak immediately in front of the ATG start and a small 53 bp synthetic polyA signal for mRNA termination. The cDNA was codon optimized for human usage and synthesized by GenScript (Piscataway, NJ). The only viral sequences included in this vector were the inverted terminal repeats of AAV2, which were required for both viral DNA replication and packaging.

The vector for this study was produced utilizing a triple-transfection method of HEK293 cells, under research grade conditions. Characterization of the vector following production included titer determination by qPCR with a supercoiled standard, endotoxin level determination (EU/mL) and a sterility assessment. The produced vector was analyzed by SDS-PAGE to verify banding pattern consistency with expected rAAV. Vector preps were titered with a linear plasmid standard and re-titered with a supercoiled plasmid standard. The vector was produced using plasmid containing the full-length human γ-sarcoglycan cDNA (NC_000013.11), a muscle specific MHCK7 promoter to drive expression, a consensus Kozak sequence (CCACC), an SV40 chimeric intron, synthetic polyadenylation site (53 bp) (FIG. 1). The SGCG expression cassette is cloned between AAV2 ITRs packaged into a self-complementary (sc) AAVrh.74 vector for enhanced transduction of cardiac tissue.

An overview of the study design is provided in TABLE 2. The dose values are determined by qPCR estimation of total number of vector genomes (vg). Inclusion of at least some partially full AAV capsids in a vector preparation may result in overestimation of dose by qPCR methods. Therefore determination of efficacy at a given dose (e.g. 5E+13) suggests efficacy may be observed at a lower qPCR-measured dose of vector when the vector is purified to remove partially full AAV capsids. The total dose (vg) and dose in terms of vector genomes per kilogram of subject (vg/kg) listed in Table 2 and throughout the Examples do not account for partially full AAV capsids.

TABLE 2
Overview of scAAVrh74.MHCK7.hSGCG Study Design

| | | | | | | Treatment | |
|---|---|---|---|---|---|---|---|
| | Delivery | Animal | Total Dose | Dose | | Endpoint | |
| Study Arm | Route | Strain | (vg) | (vg/kg) | # Mice | (months) | Analysis |
| Potency | IM | SGCG-/- | 3E+11 | N/A | 2 | 1 | IF |
| Potency | IV | SGCG-/- | 1E+13 | 5E+14 | 1 | 1.5 | IF |
| Efficacy | IV | SGCG-/- | 1E+12 | 5E+13 | 6 | 3 | IF, H&E, |
| | | | | | | | Western |
| | | | | | | | Blot, TA |
| | | | | | | | Phys, Dia |
| | | | | | | | Phys, |
| | | | | | | | Activity |
| | | | | | | | Cage, |
| | | | | | | | Histopath, |
| | | | | | | | Biodistribution |
| | | | | | | | qPCR, |
| | | | | | | | Serum |
| | | | | | | | Chem |
| Efficacy | IV | SGCG-/- | 4E+12 | 2E+14 | 6 | 3 | IF, H&E, |
| | | | | | | | Western |
| | | | | | | | Blot, TA |
| | | | | | | | Phys, Dia |
| | | | | | | | Phys, |
| | | | | | | | Activity |
| | | | | | | | Cage, |
| | | | | | | | Histopath, |
| | | | | | | | Biodistribution |
| | | | | | | | qPCR, |
| | | | | | | | Serum |
| | | | | | | | Chem |
| Efficacy | IV | SGCG-/- | 1E+13 | 5E+14 | 5 | 3 | IF, H&E, |
| | | | | | | | Western |
| | | | | | | | Blot, TA |
| | | | | | | | Phys, Dia |
| | | | | | | | Phys, |
| | | | | | | | Activity |
| | | | | | | | Cage, |
| | | | | | | | Histopath, |
| | | | | | | | Biodistribution |
| | | | | | | | qPCR, |
| | | | | | | | Serum |
| | | | | | | | Chem |
| Efficacy | IV | SGCG-/- | — | — | 6 | — | IF, H&E, |
| | | | | | | | Western |
| | | | | | | | Blot, TA |
| | | | | | | | Phys, Dia |
| | | | | | | | Phys, |
| | | | | | | | Activity |
| | | | | | | | Cage, |
| | | | | | | | Histopath, |
| | | | | | | | Serum |
| | | | | | | | Chem |
| Efficacy | IV | C57BL/6 | LRS | — | 6 | 3 | IF, H&E, |
| | | | | | | | Western |
| | | | | | | | Blot, TA |
| | | | | | | | Phys, Dia |
| | | | | | | | Phys, |
| | | | | | | | Activity |
| | | | | | | | Cage, |
| | | | | | | | Histopath, |
| | | | | | | | Serum |
| | | | | | | | Chem |

N/A: Not Applicable

IF: immunofluoresence; H&E: hematoxylin & eosin staining; TA Phys: specific force measurements and resistance to ECC injury in TA muscle; Dia Phys: specific force measurements in diaphragm muscle; Histopath: formal histopathology review; ‘−’: uninjected.

All efficacy animals were treated at 4-8 weeks of age and necropsied 3 months post-injection. SGCG−/− negative control mice were necropsied at 4 months of age.

Potency determination of the scAAVrh.74.MHCK7.hSGCG test article was achieved by performing intramuscular and systemic injections of the vector into SGCG−/− mice. Wild type mice injected with Lactated Ringer's Solution (LRS) serve as a positive control and uninjected SGCG−/− mice serve as a negative control.

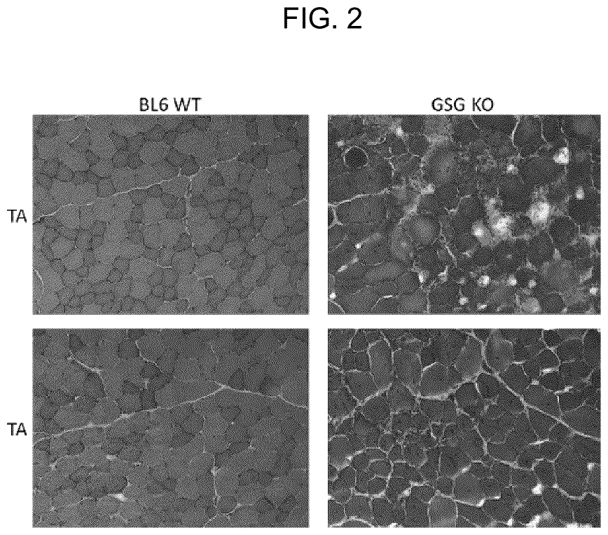

Tibialis anterior (TA) muscle from 8 week old BL6 wild-type (WT) mice and γ-sarcoglycan knockout (γ-SG KO) mice was extracted and tissue sections were stained with Hematoxylin and Eosin (H&E) to view the histology of each muscle. Even at this very early age, γ-SG KO mice demonstrated a disease phenotype in the muscle with necrotic muscle fibers, inflammatory infiltrates, and the presence of fibrotic tissue. (FIG. 2).

The SGCG AAV construct was packaged into an AAV of rh.74 serotype to create the recombinant AAV (rAAV) termed scAAVrh.74.MHCK7.hSGCG. A total of 3 mice were injected to determine potency of scAAVrh.74.MHCK7.hSGCG. One C57BL/6 WT mouse injected with LRS and one uninjected SGCG−/− mouse served as the Positive and Negative Controls respectively. The remaining 3 mice were SGCG−/− and were injected via either IM in the LTA (n=2) or IV in the tail vein (n=1) with scAAVrh.74.MHCK7.hSGCG to determine if the vector lot is potent. The study design is summarized in TABLE 3.

TABLE 3
scAAVrh.74.MHCK7.hSGCG Potency Assay

| Number | | | | | |
|---|---|---|---|---|---|
| of | Mouse | Injection | Dose | Delivery | Volume |
| Mice | Strain | Material | (vg total) | Route | (μL) |
| 1 | SGCG−/− | N/A | Negative | N/A | N/A |
| | | | Control | | |
| 1 | C57BL/6 | LRS | Positive | IV | 200 |
| | | | Control | | |
| 2 | SGCG−/− | AAV.hSGCG | 3 × 10¹¹ vg | IM | 30 |
| 1 | SGCG−/− | AAV.hSGCG | 1 × 10¹³ vg | IV | 460 |
| | | | | | (230/230) |

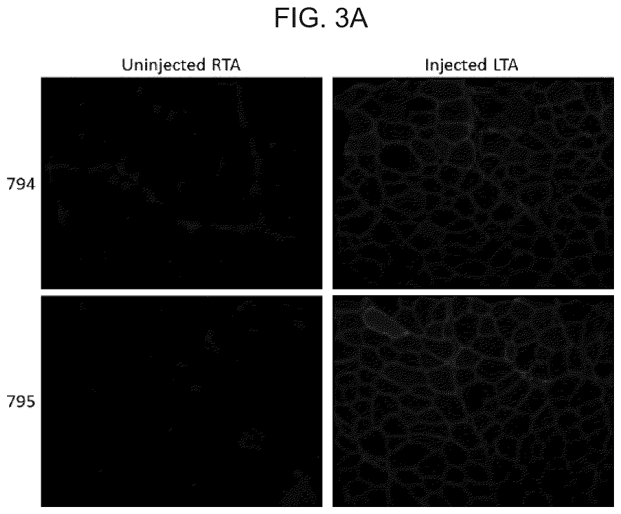

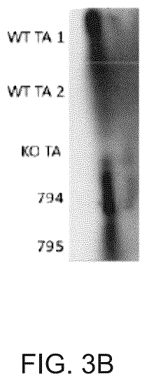

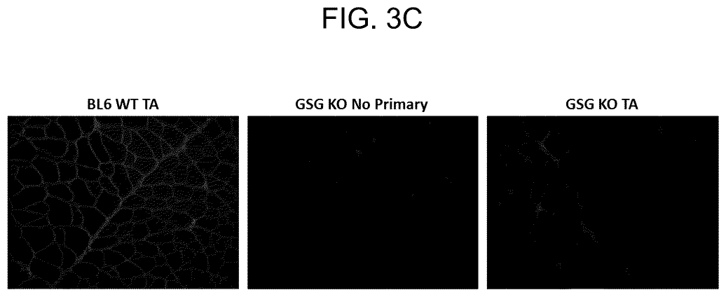

γ-SG KO mice were injected via intramuscular (IM) injection into the TA muscle at 4 weeks of age with scAAVrh.74.MHCK7.hSGCG at a dose of 3e10 vg total dose. Mice were euthanized at 4 weeks post-injection (8 weeks of age) and the TA muscle was extracted and fresh frozen in liquid nitrogen cooled methylbutane. Immunofluorescence (IF) staining for γ-sarcoglycan showed the absence of γ-sarcoglycan in uninjected right TA (RTA) muscle and showed nearly full restoration of membrane γ-sarcoglycan protein expression in injected left TA (LTA) muscle (FIG. 3A). Western blotting for γ-sarcoglycan (FIG. 3B) showed γ-sarcoglycan expression in two BL6 WT TA muscles; the absence of the protein in γ-SG KO TA muscle; and rrestoration of γ-sarcoglycan protein expression in TA muscle from injected mice #794 and #795.

Delivery of scAAVrh.74.MHCK7.hSGCG via IM to SGCG−/− mice at the specified dose of 3×10¹¹ vg total dose resulted in 93.03% expression of hSGCG in the injected LTA muscles, which is similar to the levels of our previously studied β-sarcoglycan (scAAVrh.74.MHCK7.hSGCB) vector. Immunofluorescence imaging of the vector dosed mice (Animals IDs: 794, 795) confirms expression of the hSGCG transgene (FIG. 3A). 20× images are included to visualize the amount of expression in injected muscle. As expected, the C57BL/6 WT mouse showed 100% expression of γ-sarcoglycan protein and the SGCG−/− mouse was completely absent for γ-sarcoglycan expression (FIG. 3C).

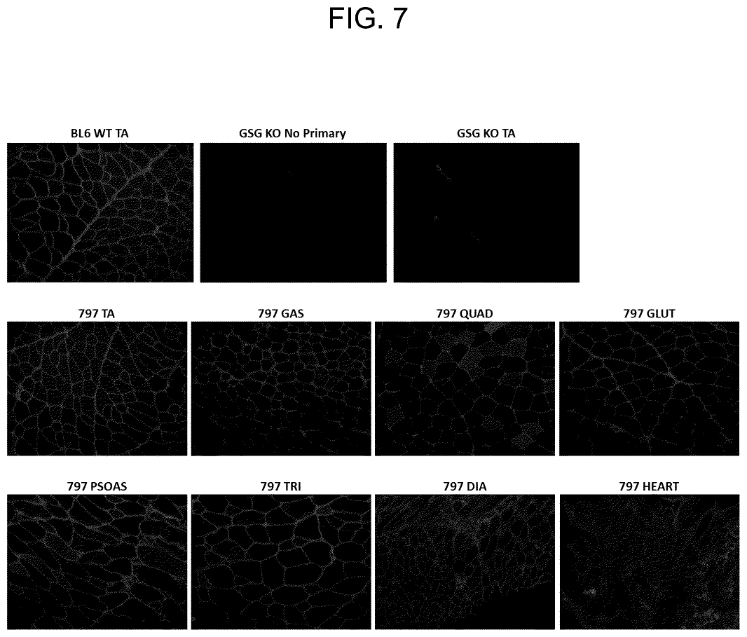

Systemic injection through the tail vein to one SGCG−/− mouse (#797) resulted in high levels of expression of the hSGCG transgene. Applicant was able to accomplish ≥94.00% transduction in all skeletal muscles of this potency mouse treated with 1×10¹³ vg total dose (5×10¹⁴ vg/kg) scAAVrh.74.MHCK7.hSGCG. The average percent expression of the AAV delivered hSGCG transgene across all skeletal muscles analyzed was 95.98%. Applicant was also able to achieve very high levels of transduction in heart upon systemic delivery. Representative 20× immunofluorescence images of all skeletal muscles along with the diaphragm and heart illustrating the widespread expression of hSGCG are shown in FIG. 7.

### Example 2: Potency and Toxicity of scAAVrh74.tMCK.hSGCB Vector in BL6 WT Mice

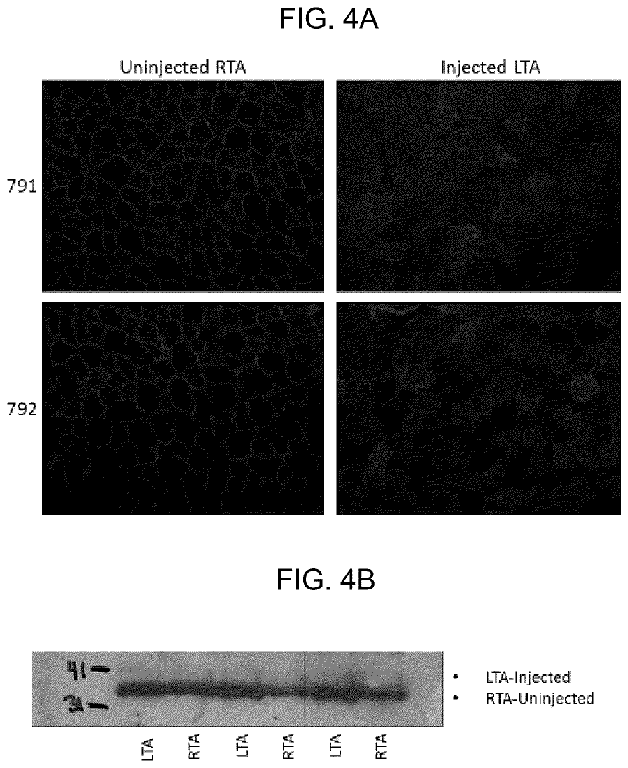

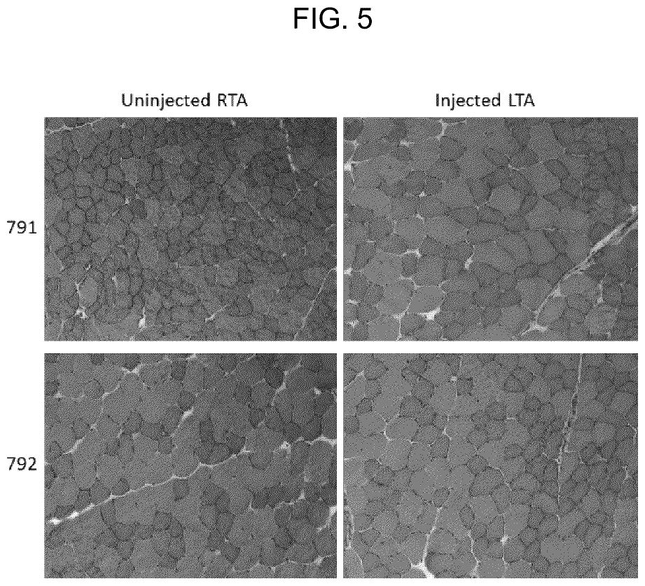

BL6 WT mice were injected via intramuscular (IM) injection into the TA muscle at 4 weeks of age with scAAVrh.74.MHCK7.hSGCG at a dose of 3e10 vg total dose. Mice were euthanized at 4 weeks post-injection (8 weeks of age) and the TA muscle was extracted and fresh frozen in liquid nitrogen cooled methylbutane. Immunofluorescence (IF) staining for γ-sarcoglycan showed the membrane staining for γ-sarcoglycan in uninjected right TA (RTA) muscle and showed intracellular staining indicative of overexpression of γ-sarcoglycan protein in injected left TA (LTA) muscle (FIG. 4A). Western blotting for γ-sarcoglycan (FIG. 4B) showed overexpression of the γ-sarcoglycan protein in the injected LTA muscle. H&E staining of TA muscle showed no toxicity with complete absence of any central nuclei, necrotic fibers, inflammatory infiltration, or fibrotic tissue in either uninjected RTA or injected LTA. (FIG. 5).

### Example 3: Gene Expression after Systemic Delivery of scAAVrh.74.tMCK.hSGCB

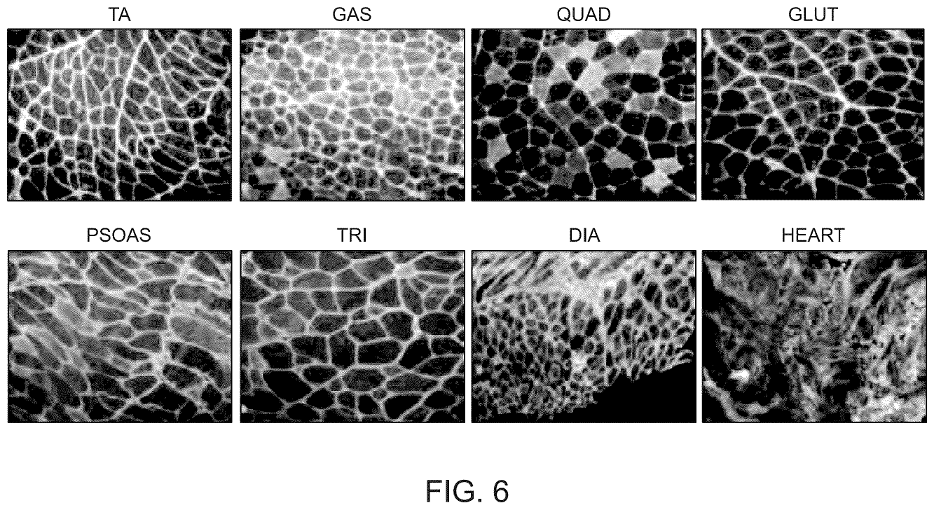

γ-SG KO mice were injected intravenously in the tail vein at 4-5 weeks of age with 1e12 vg total dose (5e13 vg/kg). Mice were euthanized after 6 weeks of treatment. Immunofluorescence staining on TA, gastrocnemius (GAS), quadriceps (QUAD), gluteus (GLUT), PSOAS, TRICEP, diaphragm, and heart muscle demonstrated widespread expression of γ-sarcoglycan (FIG. 6).

Efficacy determination of the scAAVrh.74.MHCK7.hSGCG test article was achieved by performing systemic injections in SGCG−/− mice (genotype: sgcgC57) using a single dose (1×10¹³ vg total dose, 5×10¹⁴ vg/kg). Systemic injection of scAAVrh.74.MHCK7.hSGCG at clinical dose (1×10¹² vg total dose (5×10¹³ vg/kg)), mid dose (4×10¹² vg total dose (2×10¹⁴ vg/kg)), and high dose (1×10¹³ vg total dose (5×10¹⁴ vg/kg)) into the tail vein of SGCG−/− mouse with euthanasia 3 months post-injection.

Following the results of our scAAVrh.74.MHCK7.hSGCG potency assay, Applicant delivered vector through a tail vein injection to 5 SGCG−/− mice at our potency dose of 1×10¹³ vg total dose (5×10¹⁴ vg/kg) to assess transgene expression and efficacy of our vector when delivered systemically at an extended time point of 3 months. Mice were injected at 4 weeks of age and a full necropsy was performed at 3 months post-injection. All skeletal muscles discussed above in the potency assay along with the diaphragm and heart were extracted for analysis. Organs including the lungs, kidneys, liver, spleen, and gonads were also removed for toxicology and biodistribution studies. In short, hSGCG transgene expression remained high following 3 months treatment and all muscles from treated mice were again highly transduced. This was accompanied by improved muscle histopathology and improved TA and diaphragm muscle function. Systemic delivery of the scAAVrh.74.MHCK7.hSGCG vector did not induce any toxicity in muscles or organs

### γ-Sarcoglycan Expression

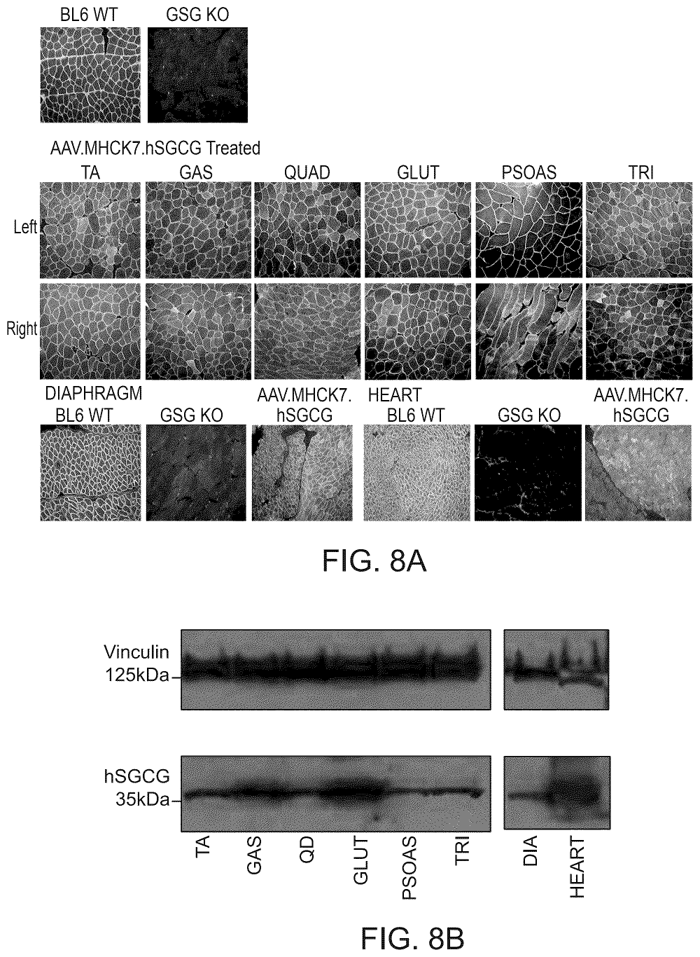

Immunofluorescence staining for human γ-sarcoglycan was used to determine hSGCG transgene expression in six skeletal muscles, both left and right, in additional to the diaphragm and heart of all the SGCG−/− mice given a systemic injection of the scAAVrh.74.MHCK7.hSGCG vector. These muscles included the TA, GAS, QUAD, GLUT, PSOAS, TRI. For the purposes of expression analysis and transduction efficiency, images for the left and right muscles from 5 treated mice were utilized for quantification. Four 20× images were taken of each muscle and the percent of hSGCG positive fibers (number of positive expressing fibers/total number of fibers) was determined for each image resulting in the average percent transduction for each muscle from each mouse. FIG. 8A shows representative images from the treated mice and demonstrate the high levels of expression averaging 92.26% across all muscles quantified including the diaphragm. Applicant again also saw high levels of transduction in cardiac muscle in all vector treated mice. FIG. 8B shows Western blotting that confirms expression of the hSGCG transgene in all skeletal muscles and the heart from mice given intravenous delivery of the scAAVrh.74.MHCK7.hSGCG vector. TABLE 4 lists the average percent expression among the four 20× images for each muscle from each mouse, along with the average for each muscle across all 5 mice.

TABLE 4
Average Percent γ-Sarcoglycan Transgene Expression

| Animal ID | 5229 | 5230 | 5231 | 5232 | 5233 | Average |
|---|---|---|---|---|---|---|
| Muscle | | | | | | |
| TA | 97.79 | 97.79 | 99.54 | 99.13 | 96.59 | 98.17 |
| GAS | 90.88 | 90.88 | 74.14 | 97.67 | 93.49 | 89.41 |
| QUAD | 88.37 | 88.37 | 96.85 | 95.01 | 76.15 | 88.95 |
| GLUT | 96.19 | 96.19 | 91.12 | 100 | 92.83 | 95.27 |
| PSOAS | 97.55 | 97.55 | 99.01 | 49.88 | 97.92 | 88.38 |
| TRI | 97.32 | 97.32 | 99.31 | 94.98 | 93.31 | 96.45 |
| DIA | 93.02 | 93.02 | 75.56 | 97.14 | 87.22 | 89.19 |

### Histopathology of Treated Muscle

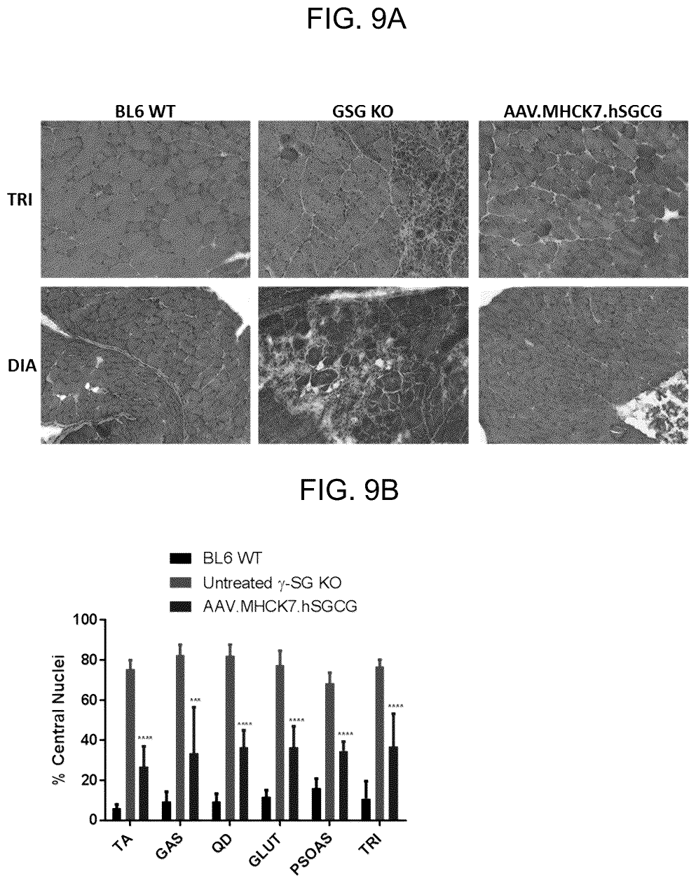

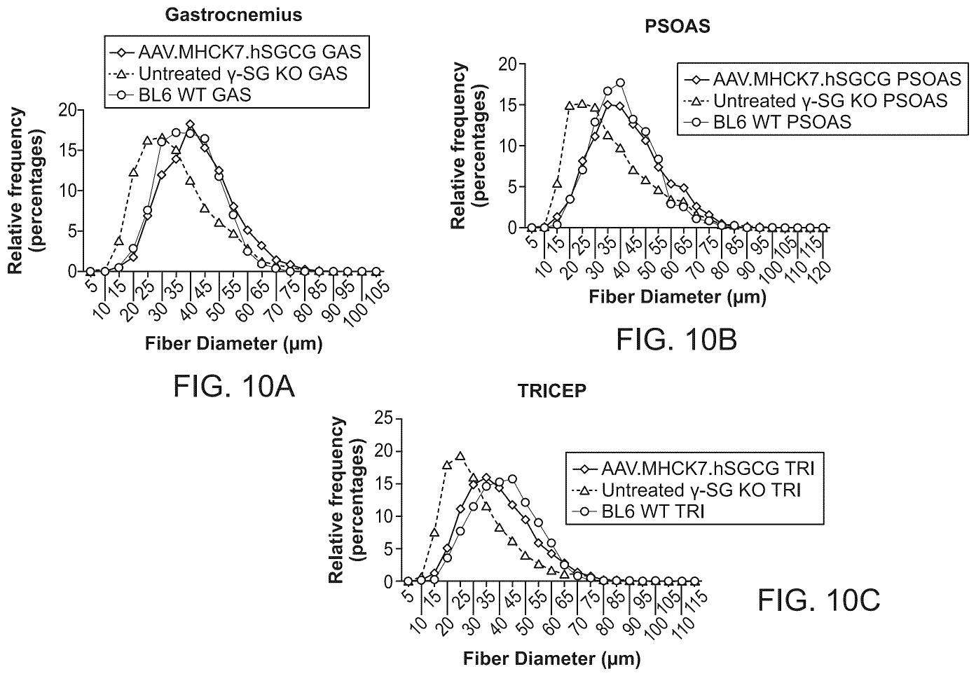

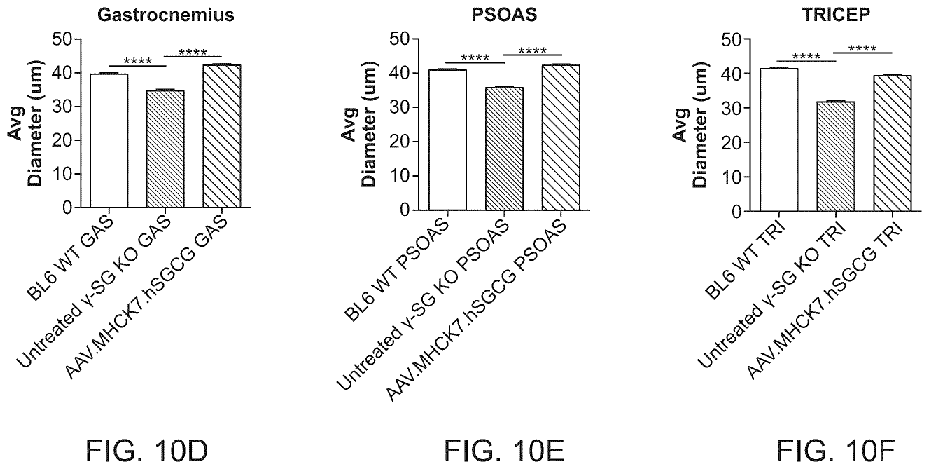

Muscles from SGCG−/− mice, both skeletal and cardiac, exhibit widespread myopathy including pronounced myofiber atrophy and hypertrophy with multiple focal areas of necrosis. Also present are increasing numbers of mononuclear cell inflammation (lymphocytes and macrophages, with scattered neutrophils) and increased dystrophic calcification, fatty infiltration, central nucleation, and fibrosis. Hematoxylin & eosin staining in FIG. 9A illustrates this dystrophic phenotype in SGCG−/− mice when compared to normal WT mice and the improvement of muscle pathology following treatment. Quantification of histological parameters shows a significant elevation of the number of centrally nucleated fibers in the skeletal muscles of SGCG−/− mice, followed by a reduction in central nucleation in numerous different skeletal muscles as a result of γ-sarcoglycan gene transfer (FIG. 9B). A more in-depth analysis of muscle histopathology reveals a normalization of fiber size distribution accompanied by an increase in average fiber diameter in diseased SGCG−/− mice treated with vector in all three muscles examined (GAS, PSOAS, and TRI) (FIGS. 10A-10F). The individual central nuclei counts and average fiber diameters for the various muscles were analyzed from each mouse.

### Example 4: Physiological Deficits of γ-SG KO Mice

Sirius red stain will be performed to quantify the amount of fibrotic tissue. γ-SG KO mice and BL6 WT mice will be tested at 4 months of age to assess whether there are force deficits in skeletal muscle. The tibialis anterior (TA) muscle will be tested for a significant decrease in specific force and resistance to injury compared to controls. The diaphragm muscle will also be tested in a similar manner to detect any significant decreases. This measurable decrease will provide a functional outcome measure to establish efficacy for AAV.hSGCB therapy.

### Example 5: Functional Outcomes after scAAVrh74.tMCK.hSGCB Treatment

Cohorts of γ-SG KO mice will be injected on a rolling basis for three-month studies to quantify efficacy and toxicity (TABLE 5). Mice will be subjected to activity cage analysis prior to euthanasia to determine overall activity of treated mice compared to γ-SG KO controls. TA and diaphragm muscle will be subjected to physiology analysis to determine specific force outputs and resistance to injury/fatigue. γ-SG KO muscles will be compared to BL6 WT controls to establish functional outcome measures that will be used to determine efficacy of treatment in treated mice. All skeletal muscles will be IF (immunofluorescence) stained for expression of γ-sarcoglycan, H&E stained for histopathology. Quantitative polymerase chain reaction (qPCR) will be performed on muscles and organs from injected mice to determine vector genome biodistribution.

TABLE 5

| | | | Sample | |
|---|---|---|---|---|
| Mouse Strain | Test Article | Human Dose | Size | Endpoint |
| C57/BL6 | LR* | NA | 6 | 12 weeks |
| SGCG KO | LR | NA | 6 | 12 weeks |
| SGCG KO | scAAVrh74.MHCK7.SGCG | 5 × 10¹³ vg/kg | 6 | 12 weeks |
| SGCG KO | scAAVrh74.MHCK7.SGCG | 1 × 10¹⁴ vg/kg | 6 | 12 weeks |
| SGCG KO | scAAVrh74.MHCK7.SGCG | 2 × 10¹⁴ vg/kg | 6 | 12 weeks |

### Example 6: Functional Assessment of Systemic Delivery

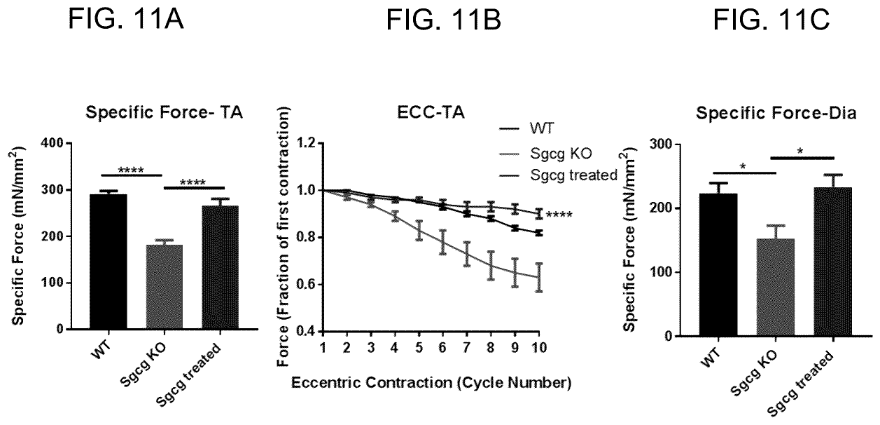

To determine whether hSGCG gene transfer provides a functional benefit to diseased muscle, Applicant assessed the functional properties of the TA and diaphragm muscle from SGCG−/− mice treated with scAAVrh.74.MHCK7.hSCGG. As outlined in Examples 1 to 5, Applicant first demonstrated histopathology in limb skeletal muscle and the diaphragms in mice in the absence of γ-sarcoglycan. In situ analysis of the TA muscle of untreated SGCG−/− mice revealed a statistically significant decrease of 37.68% in normalized specific force production compared to BL6 WT TA muscles (BL6 WT: 291.65 mN/mm² vs. SGCG−/−: 181.77 mN/mm²). Specific force outputs were significantly increased to normal WT levels compared to SGCG−/− muscle following treatment (SGCG−/−: 181.77 mN/mm² vs. Treated: 266.02 mN/mm²) (FIG. 11A and FIG. 11C). One additional functional outcome measure to determine the functional benefits of hSGCG gene transfer is to assess the resistance to contraction induced injury in the TA muscle following repeated eccentric contractions. The TA muscle of normal BL6 WT mice untreated SGCG−/− mice lost only 18% of force production following a round of 10 eccentric contractions, compared to a 37% loss of force in untreated SGCG−/− TA muscle. Vector treated SGCG−/− muscles had an improvement to above WT levels where they saw only a 10% loss of force following the eccentric contraction (ECC) protocol (FIG. 11B).

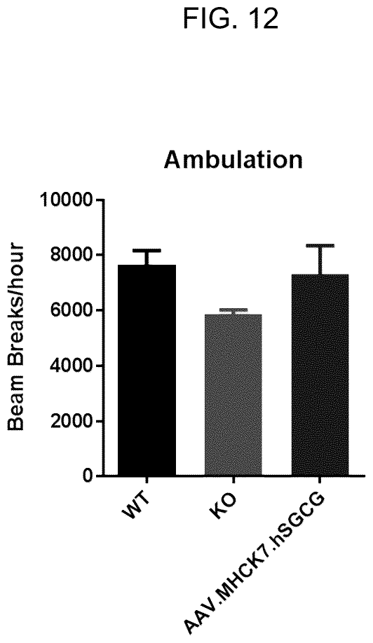

In order to further test potential functional benefits resulting from a systemic delivery of a therapeutic hSGCG transgene and ultimately improving the disease phenotype of SGCG−/− mice, laser-monitoring of open-field cage activity was performed on all groups of mice. The graph in FIG. 12 depicts a decrease of 23.64% in total ambulation in x and y planes in SGCG−/− mice compared to normal BL6 WT (BL6 WT: 7655.42 beam breaks/hr vs. SGCG−/−: 5846.00 beam breaks/hr). scAAVrh.74.MHCK7.hSGCG treated mice were overall more active compared to SGCG−/− mice by qualitative observation, and a quantitative measurement of the open-field cage activity showed a 24.90% increase in ambulation (SGCG−/−: 5846.00 beam breaks/hr vs Treated: 7301.80 beam breaks/hr). The detailed values for each parameter in individual mice were also measured.

### Example 7: Toxicology and Vector Biodistribution

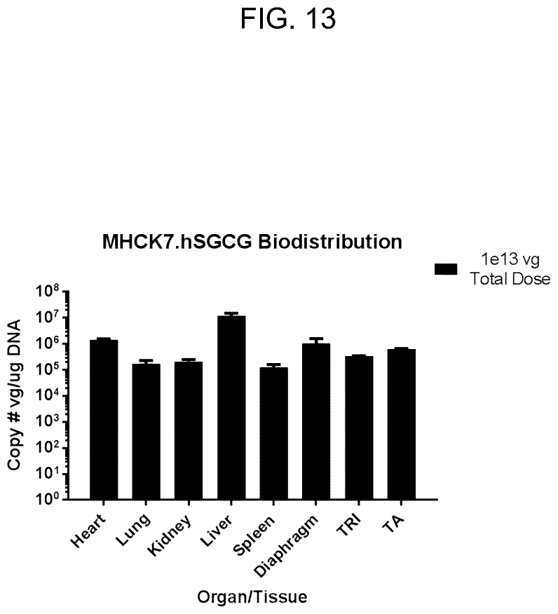

The purpose of this study was to assess any potential toxicity or safety concerns of hSGCG gene therapy in SGCG−/− mice at 3 months after delivery of the test article scAAVrh.74.MHCK7.hSGCG, utilizing the same animals described above. Test article was given to 5 SGCG−/− at 1.0×10¹³ vg total dose (5×10¹⁴ vg/kg) by the intravenous (IV) route in a volume of 460 μL split into two separate 230 μL injections, morning and afternoon, at 4 weeks of age. Six uninjected SGCG−/− mice served as untreated diseased controls, and 5 C57BL/6 WT mice served as normal healthy controls (TABLE 6). Full necropsies were performed on all mice to extract six skeletal muscles (TA, GAS, QUAD, GLUT, PSOAS, and TRI), both left and right side, along with the diaphragm and heart, as well as internal organs including the lungs, kidneys, liver, spleen, and gonads. To assess the safety of our vector, hematoxylin & eosin staining was performed on cryosections of the muscle tissue and all organs harvested were formalin fixed and also stained with hematoxylin & eosin. These sections were then formally reviewed for toxicity by an independent veterinary pathologist and no adverse effects were detected, and the results are summarized below in TABLE 7 and the detailed histopathology report was also prepared. Quantitative PCR was performed to assess vector biodistribution and those results are shown below in TABLE 7 and FIG. 13.

### Histopathology Review of Vector Transduced Tissue

In order to determine the safety and toxicology profile of scAAVrh.74.MHCK7.hSGCG using systemic delivery, all skeletal muscles including the diaphragm, along with the heart and five other organs harvested from the group of vector dosed SGCG−/− mice and controls from this pre-clinical study were stained with H&E and sections of each tissue were formally reviewed by an independent veterinary pathologist. Group details and study design are shown in TABLE 6.

TABLE 6
Summary of Cohorts for scAAVrh.74.MHCK7.hSGCG Gene Transfer
Histopathology Review

| | | | | | Age at | | Time on |
|---|---|---|---|---|---|---|---|
| | | | Dose | | In- | Age at | Treat- |
| | Genotype | Cohort | (vg) | Sex | jection | Necropsy | ment |
| 1 | SGCG-/- | Test | 1.0 × | Female | 1 month | 4 months | 3 months |
| | | Article | 10¹³ | Female | 1 month | 4 months | 3 months |
| | | | | Female | 1 month | 4 months | 3 months |
| | | | | Female | 1 month | 4 months | 3 months |
| | | | | Female | 1 month | 4 months | 3 months |
| 2 | BL6 WT | Vehicle | LRS | Male | 1 month | 4 months | 3 months |
| | | Control | | Male | 1 month | 4 months | 3 months |
| | | | | Male | 1 month | 4 months | 3 months |
| | | | | Male | 1 month | 4 months | 3 months |
| | | | | Male | 1 month | 4 months | 3 months |
| 3 | SGCG-/- | Disease | N/A | Female | N/A | 4 months | N/A |
| | | Control | | Female | N/A | 4 months | N/A |
| | | | | Male | N/A | 4 months | N/A |
| | | | | Male | N/A | 4 months | N/A |

In summary, IV injection of scAAVrh.74.MHCK7.hSGCG did not elicit any microscopic changes in myofibers of any skeletal muscles examined (TABLE 7). In addition, no treatment-related lesions were seen in any of the tissues evaluated histologically, indicating the test article was well tolerated, see full report in Appendix J (Report No. AAVrh74-SGCG-MOUSE-001.1). Any changes noted were seen in both treated and control mice and were considered incidental findings. Furthermore, the independent review indicated that relative to reference specimens from control mice, administration of the test article scAAVrh.74.MHCK7.hSGCG substantially reduced myofiber atrophy, degeneration, and destruction, suggesting the vector can ameliorate the degree of myopathy associated with the absence of SGCG in diseased mice.

TABLE 7
Histopathology Results scAAVrh.74.MHCK7.hSGCG Safety Study in SGCG−/− Mice

| Test Article | Age at | Treatment | Tissues | Formal |
|---|---|---|---|---|
| (vector dose) | Injection | Length | Analyzed | Histopath |
| scAAVrh.74.MHCK7.hSGCG | 1 month | 3 months | Skeletal | No Findings |
| (1e13vg total dose-5e14 vg/kg) | | | Muscles, Heart, | |
| 5 animals analyzed | | | Lungs, Kidney, | |
| | | | Liver, Spleen, | |
| | | | Gonads | |

### Vector Genome Biodistribution

The presence of test article-specific DNA sequences was examined using a real time, quantitative PCR assay (qPCR). Biodistribution analysis was performed on tissue samples collected from two vector dosed SGCG−/− animals. A positive signal is anything equal to or greater than 100 single-stranded DNA copies/μg genomic DNA detected. Tissues were harvested at necropsy and vector specific primer probe sets specific for sequences of the MHCK7 promoter were utilized. TABLE 8 and FIG. 13 depict the vector genome copies detected in each tissue sample from scAAVrh.74.MHCK7.hSGCG injected mice.

scAAVrh.74.MHCK7.hSGCG transcript was detected at varying levels in all collected tissues. As expected, while vector was detected at high levels in the liver due to the nature of the intravenous delivery route, the highest levels were seen in skeletal muscle and the heart. The lowest levels were detected in the lungs, kidney, and spleen. These data indicate that the test article was efficiently delivered into all investigated tissues of vector dosed mice.

TABLE 8
Quantitative PCR Results Following
High Dose scAAVrh.74.MHCK7.hSGCG
Systemic Delivery in SGCG-/- Mice

| | Vector genome copies/μg | |
|---|---|---|
| Tissue | 5229 | 5231 |
| Heart | 1.56E+06 | 1.30E+06 |
| Lung | 1.15E+05 | 2.29E+05 |
| Kidney | 1.74E+05 | 2.48E+05 |
| Liver | 9.23E+06 | 1.50E+07 |
| Spleen | 1.58E+05 | 9.05E+04 |
| Diaphragm | 4.63E+05 | 1.60E+06 |
| TRI | 3.35E+05 | 3.48E+05 |
| TA | 6.23E+05 | 6.70E+05 |

### Analysis of Serum Chemistries

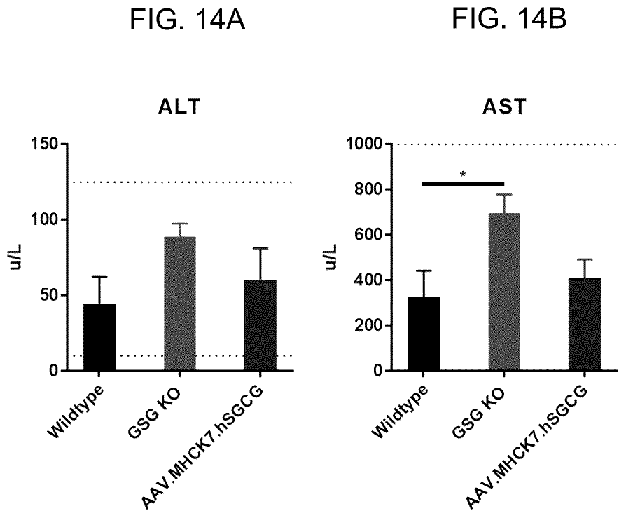

To further evaluate liver function, Applicant assessed the levels of two liver enzymes that are normal serum chemistry parameters, alkaline aminotransferase (ALT) and aspartate aminotransferase (AST). Elevation of either of these enzymes can be indicative of hepatocyte damage and impaired liver function. Applicant analyzed the serum from all 6 C57BL/6 WT mice, all 6 untreated SGCG−/− mice, and all 5 scAAVrh.74.MHCK7.hSGCG dosed mice. FIG. 14A show an elevation of ALT in untreated SGCG−/− mice to double the levels seen in healthy BL6 WT mice (BL6 WT: 44.20 U/L vs. SGCG−/−: 89.00 U/L). IV delivery of the scAAVrh.74.MHCK7.hSGCG to SGCG−/− mice resulted in a 32.02% decrease in ALT levels (SGCG−/−: 89.00 U/L vs. Treated: 60.50 U/L). FIG. 14B shows AST levels in all three groups of mice indicated a significant elevation of 113.27% in untreated SGCG−/− mice (BL6 WT: 326.00 U/L vs. SGCG−/−: 695.25 U/L). These AST levels were reduced by 41.10% following systemic delivery of scAAVrh.74.MHCK7.hSGCG (FIG. 14B). Taken together, while liver enzymes considered to be biomarkers of liver damage are elevated in diseased SGCG−/− mice, systemic hSGCG gene transfer in SGCG−/− diseased mice normalizes the levels of both ALT and AST. Individual values for each enzyme in all mice were determined.

In conclusion, systemic delivery of two different doses of the AAV virus carrying the hSGCB transgene was shown to be safe and non-toxic. Doses tested include 1.2×10¹³ vg total dose (6.0×10¹⁴ vg/kg) and 1.0×10¹³ vg total dose (5.0×10¹⁴ vg/kg). In particular, systemic delivery of a high dose (1.0×10¹³ vg total dose −5.0×10¹⁴ vg/kg) of scAAVrh.74.MHCK7.hSGCG through the tail vein of SGCG−/− is safe and effective in restoring γ-sarcoglycan expression and reversing dystrophic histopathology in diseased muscle.

What is claimed:

1. A method of treating γ-sarcoglycanopathy in a subject, comprising administering to the subject a therapeutically effective amount of a composition comprising a polynucleotide sequence encoding γ-sarcoglycan.

2. The method of claim 1, wherein the polynucleotide sequence encodes an amino acid sequence as set forth in SEQ ID NO: 2.

3. The method of claim 1, wherein the polynucleotide sequence encoding γ-sarcoglycan is codon optimized.

4. The method of claim 1, wherein the polynucleotide sequence encoding γ-sarcoglycan comprises SEQ ID NO: 1.

5. The method of claim 1, where the polynucleotide sequence further comprises an AAV vector.

6. The method of claim 1, wherein the polynucleotide encoding γ-sarcoglycan is operatively linked to a muscle-specific control element selected from the group consisting of human skeletal actin gene element, cardiac actin gene element, myocyte-specific enhancer binding factor (mef) element, muscle creatine kinase (MCK) promoter, truncated MCK (tMCK) promoter, myosin heavy chain (MHC) element, MHCK7 promoter, C5-12, murine creatine kinase enhancer element, skeletal fast-twitch troponin c gene element, slow-twitch cardiac troponin c gene element, the slow-twitch troponin I gene element, hypoxia-inducible nuclear factors, steroid-inducible element, and glucocorticoid response element (gre).

7. The method of claim 1, wherein the method further comprises administering to the subject a corticosteroid.

8. The method of claim 7, wherein the polynucleotide and corticosteroid are administered concurrently or sequentially.

9. The method of claim 7, wherein the corticosteroid is administered prior to the polynucleotide.

10. A polynucleotide comprising a nucleotide sequence encoding γ-sarcoglycan, wherein the nucleotide sequence comprises a nucleotide sequence at least 95% identical to SEQ ID NO: 1.

11. The polynucleotide of claim 10, wherein the nucleotide encoding γ-sarcoglycan is operatively linked to a muscle-specific control element selected from the group consisting of human skeletal actin gene element, cardiac actin gene element, myocyte-specific enhancer binding factor (mef) element, muscle creatine kinase (MCK) promoter, truncated MCK (tMCK) promoter, myosin heavy chain (MHC) element, MHCK7 promoter, C5-12, murine creatine kinase enhancer element, skeletal fast-twitch troponin c gene element, slow-twitch cardiac troponin c gene element, the slow-twitch troponin I gene element, hypoxia-inducible nuclear factors, steroid-inducible element, and glucocorticoid response element (gre).

12. The polynucleotide of claim 10, wherein the nucleotide sequence encodes the amino acid sequence of SEQ ID NO: 2.

13. A method of treating γ-sarcoglycanopathy in a subject, comprising administering to the subject a therapeutically effective amount of a composition comprising the polynucleotide of claim 10.

14. The method of claim 13, wherein the composition further comprises a pharmaceutically acceptable carrier.

15. A host cell comprising the polynucleotide of claim 10.

16. A combination therapy comprising administering to a subject in need thereof a therapeutically effective amount of the polynucleotide of claim 10 and a corticosteroid.

17. The combination therapy of claim 16, wherein the polynucleotide and corticosteroid are administered concurrently.

18. The combination therapy of claim 16, wherein the polynucleotide and corticosteroid are administered sequentially.

19. The combination therapy of claim 16, wherein the corticosteroid is administered prior to the polynucleotide.

20. A kit, comprising the polynucleotide of claim 10 and a corticosteroid.
